(19) United States
(12) Patent Application Publication
(10) Pub. No.: US 2006/0104969 A1
(43) Pub. Date: May 18, 2006

(54) Compositions and methods for enhancing structural and functional nervous system reorganization and recovery

(75) Inventors: Serkan Oray, Cambridge, MA (US); Ania K. Majewska, Honeoye Falls, NY (US); Mriganka Sur, Cambridge, MA (US); Yang Teng, Wellesley, MA (US)

Correspondence Address: CHOATE, HALL & STEWART LLP, TWO INTERNATIONAL PLACE, BOSTON, MA, 02110, US

(73) Assignees: Massachusetts Institute of Technology; Brigham & Women's Hospital, Inc.

(21) Appl. No.: 11/205,501

(22) Filed: Aug. 16, 2005

Related U.S. Application Data
(60) Provisional application No. 60/601,863, filed on Aug. 16, 2004.

(51) Int. Cl. A61K 38/48

(52) U.S. Cl. 424/94.63; 424/94.64

(57) ABSTRACT

The present invention provides methods and compositions for enhancing recovery in a subject suffering from damage to the nervous system. In particular, the invention includes a method for promoting recovery and/or reorganization in the nervous system of a subject in need of enhancement of recovery and/or reorganization of the nervous system as a result of ischemic, hemorrhagic, neoplastic, degenerative, or traumatic damage by focally administering a composition comprising a proteolysis-enhancing agent such as tissue plasminogen activator (tPA), plasmin, or a PAI inhibitor to the nervous system of the subject. In some embodiments an additional active agent is also administered. The composition can be delivered using a variety of techniques including injection, via infusion pump, from an implantable microchip, or using a polymeric delivery vehicle. The composition can be administered, for example, to one or more subdivisions or areas of the brain, the spinal cord, or to one or more nerves or nerve tracts innervating diverse regions of the body. The invention also includes a drug delivery device for implantation into the nervous system to promote nervous system reorganization and/or recovery following ischemic, hemorrhagic, neoplastic, traumatic or degenerative damage, the drug delivery device comprising a biocompatible polymer and a proteolysis-enhancing agent such as tissue plasminogen activator (tPA), plasmin, or a PAI inhibitor, wherein the proteolysis-enhancing agent is released from the polymer in an amount effective to promote structural reorganization of the nervous system. In some embodiments the biocompatible polymer is a hydrogel.

### CROSS-REFERENCE TO RELATED APPLICATIONS

This application claims priority to U.S. Provisional Application No. 60/601,863 filed Aug. 16, 2004, which is herein incorporated by reference.

### BACKGROUND OF THE INVENTION

Strokes are a result of a sudden disruption of blood flow to a part of the brain, which can damage and/or kill nerve cells. A stroke occurs when a blood vessel that normally supplies brain tissue either bursts or becomes transiently or permanently blocked, such as by a blood clot (e.g., a thromboembolus) or other embolus or obstruction. The resulting disruption in normal blood flow deprives the affected tissue of needed oxygen and nutrients and can also impair removal of waste products. Ischemic strokes, caused by blood vessel blockage, are by far the most common (approximately 88%), with hemorrhagic strokes (intracerebral or subarachnoid hemorrhage) accounting for the remainder (American Heart Association. Heart Disease and Stroke Statistics—2004 Update. Dallas, Tex.: American Heart Association; 2003). Hemorrhage not only disrupts the normal function of the ruptured vessel but can also cause tissue compression, resulting in ischemia. Edema, associated tissue compression, also contributes to brain injury following ischemic stroke. The release of neurotoxic substances is also believed to contribute to nervous system damage in stroke victims.

Approximately 700,000 people suffer a first or recurrent stroke annually in the United States, resulting in over 150,000 deaths. Currently the only therapy for ischemic stroke approved by the Food and Drug Administration (FDA) is infusion of the thrombolytic agent tissue type plasminogen activator (tPA) within a short time window following the causative event. Such thrombolytic therapy was shown to be both safe and beneficial if delivered within a 3 hours of the onset of symptoms (NINDS, Tissue plasminogen activator for acute ischemic stroke. The national institute of neurological disorders and stroke RT-PA stroke study group. N. Engl. J. Med. 333: 1581-1587, 1995).

While stroke is the third leading cause of death in industrialized countries, in most cases stroke is not fatal. However, stroke is a major cause of morbidity and is a leading cause of serious, long-term disability. About 4.8 million stroke survivors are alive today in the United States, with a much larger total number worldwide. Many of these individuals suffer from functional limitations affecting the senses, motor activity, speech and/or the ability to understand speech, behavior, thought patterns, memory, emotions, or other aspects of cognition. Although functional deficits following stroke may be permanent, in many cases full or partial recovery is possible. The mainstays of treatment are supportive care and rehabilitation therapy, which frequently continues for months or years. Unfortunately, there are no pharmacological agents that have demonstrated efficacy in improving the long-term outcome of stroke.

Although stroke represents the most common cause of damage to the central nervous system (CNS), a number of other conditions are also significant causes of functional deficits due to loss of brain tissue, either as a direct consequence of injury, or secondary to events such as swelling. Among these are primary brain tumors, brain metastases, and surgery for these or other conditions.

Approximately 10,000-12,000 individuals suffer spinal cord injuries (SCI) each year in the United States, bringing the projected prevalence rate in the United States to nearly 280,000 by the year 2014 (DeVivo, M. J., 2002) Improvements in supportive care have greatly increased the survival rate following such injuries, but therapeutic options remain limited, and efforts focus on rehabilitation. The pathophysiology of traumatic SCI is believed to involve primary mechanical injury to the cord tisse and delayed secondary injury processes arising as a result of a number of different mechanisms (Teng & Wrathall, 1997, and references therein). Tumors affecting the spinal cord or meninges (either primary tumors or metastases) are also a significant source of morbidity. Such tumors may arise in nervous system tissue, connective tissue, etc.

It is therefore evident that a significant need in the art exists for improved treatments, particularly pharmacological treatments, that would enhance recovery following damage to the CNS. In addition, there remains a need in the art for improved treatments that would enhance recovery following damage to the peripheral nervous system (PNS) since current treatments such as nerve grafts, while often helpful, have a number of limitations.

### SUMMARY OF THE INVENTION

The present invention addresses these needs, among others. In one aspect, the invention provides a method for promoting recovery or reorganization in the nervous system of a subject comprising the step of: focally administering a composition comprising a proteolysis-enhancing agent to the central or peripheral nervous system of a subject in need of enhancement of recovery or reorganization of the nervous system. The subject typically is in need of such recovery or reorganization as a result of ischemic, hemorrhagic, neoplastic, degenerative, or traumatic damage to the nervous system, i.e., the subject has suffered such damage. The method can include a step of identifying or providing, e.g., diagnosing a subject as having suffered such damage. The method can include a step of identifying or diagnosing the subject as having a reasonable likelihood, within sound medical judgment, of benefiting from the treatment. The method can include a step of determining an appropriate route and/or location for administration of the composition. Certain of the proteolysis-enhancing agents disclosed herein can be administered by a method other than focal administration, e.g., they can be administered systemically for treatment of certain conditions.

Included in the invention are embodiments wherein the proteolysis-enhancing agent is a proteolytic agent. Also included in the invention are embodiments wherein the proteolysis-enhancing agent is a protease. In certain embodiments the proteolysis-enhancing agent is plasmin, a plasminogen activator, or an inhibitor of an endogenous plasminogen activator inhibitor. For example, the proteolysis-enhancing agent is tissue plasminogen activator (tPA), e.g., human tPA, in certain embodiments. In certain embodiments the the proteolysis-enhancing agent is plasmin. In certain embodiments the proteolysis-enhancing agent promotes degradation of a component of the extracellular matrix. In certain embodiments the proteolytic agent directly or indirectly degrades fibrin. In certain embodiments the composition is administered by implanting into the subject a drug delivery device that releases the proteolysis-enhancing agent over a period of time at or in the vicinity of a desired location. The desired location can be, for example, an area of ischemic, hemorrhagic, neoplastic, degenerative, or traumatic damage in the central or peripheral nervous system, or is an oppositely located brain hemisphere. In some embodiments the drug delivery device comprises a pump. In some embodiments the drug delivery device comprises a biocompatible polymer, e.g., a biodegradable polymer. In some embodiments the polymeric matrix of the drug delivery device is a hydrogel. In some embodiments the composition a plurality of polymeric microparticles or nanoparticles having the proteolysis-enhancing agent associated therewith (e.g., encapsulated therein, adsorbed thereon, entangled in a polymer network, etc.). Optionally the proteolysis-enhancing agent is covalently attached to the polymer by an optionally cleavable linkage. In some embodiments the proteolysis-enhancing agent is delivered in a solution that forms a gel following contact with physiological fluids. The proteolysis-enhancing agent may, for example, be delivered in an amount effective to promote structural reorganization of synaptic connections, increase formation of new synaptic connections, increase dendritic spine motility, promote growth of axons, inhibit at least in part functional and/or structural deterioration or degradation, or any combination of the foregoing.

In certain embodiments the composition further comprises an agent selected from the group consisting of neural growth enhancing agents, which are optionally selected from among neurotransmitters or analogs thereof, neurally active growth factors, neural signaling molecules, and neurally active small molecules, and neurally active metals. Alternately, one or more of these agents can be administered separately, either by focal administration to the nervous system or by an alternate route.

The invention further provides a method of treating a subject in need of enhancement of recovery or reorganization in the nervous system comprising focally administering a composition comprising a proteolysis-enhancing agent to the central or peripheral nervous system of the subject. The subject will typically have suffered nervous system damage as a result of ischemic, hemorrhagic, neoplastic, degenerative, or traumatic damage. Other features of the method for promoting recovery or reorganization in the nervous system are also applicable in this method. For example, the composition can comprise plasmin, a plasminogen activator, or an inhibitor of an endogenous plasminogen activator inhibitor.

The invention further provides a method of treating a subject in need of enhancement of recovery or reorganization in the nervous system comprising administering a composition comprising a proteolysis-enhancing agent and administering a composition comprising a neural growth enhancing agent to the subject. The proteolysis-enhancing agent can be a proteolytic agent, e.g,. a protease. Either or both of the agents can be administered to the central or peripheral nervous system. Either or both of the agents can be administered focally to the central or peripheral nervous system, e.g., to a desired location therein. The agents can be administered separately or in a single composition. Any of the methods for administration contemplated herein can be used.

In any of the methods, the subject may be engaged in a program of rehabilitation designed to promote functional recovery following ischemic, hemorrhagic, neoplastic, or traumatic damage to the nervous system, wherein the subject is so engaged during at least part of the time interval during which the agent is administered or during which the agent remains active in the nervous system of the subject.

In another aspect, the invention provides a drug delivery device comprising: a biocompatible polymer; and a proteolysis-enhancing agent, wherein the proteolysis-enhancing agent is released from the polymer in an amount effective to promote structural or functional recovery or reorganization in the nervous system of the subject. The proteolysis-enhancing agent can be, e.g., tPA, plasmin, or an inhibitor of endogenous plasminogen activator inhibitor.

In another aspect the invention provides a composition comprising a proteolysis-enhancing agent and a neural growth enhancing agent, which is optionally selected from among neurotransmitters or analogs thereof, neurally active growth factors, neural signaling molecules, and neurally active small molecules, and neurally active metals. The invention further comprises drug delivery devices, e.g., polymer-based drug delivery devices, comprising the composition.

This application refers to various patents and publications. The contents of all of these are incorporated by reference. In addition, the following publications are incorporated herein by reference: Ausubel, F., (ed.). Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Kandel, E., Schwartz, J. H., Jessell, T. M., (eds.), Principles of Neural Science, 4^{th} ed., McGraw Hill, 2000; Cowan, W. M., Südhof, T. C., and Stevens, C. F., (eds.), Synapses, The Johns Hopkins University Press, Baltimore and London, 2001; and Hardman, J., Limbird. E., Gilman, A. (Eds.), Victor, M. and Ropper, A. H., Adams and Victor's Principles of Neurology, 7^{th} ed., McGraw Hill, 2000; Grossman, R. I. and Yousem, D. M., Neuroradiology: The Requisites, 2^{nd} ed., C. V. Mosby, 2003; Gillen, G. and Burkhardt, A. (eds.), Stroke Rehabilitation: A Function-Based Approach, 2^{nd} ed., C. V. Mosby, 2004; Somers, M. F., Spinal Cord Injury: Functional Rehabilitation, 2^{nd} ed., Prentice Hall, 2001; Goodman and Gilman's The Pharmacological Basis of Therapeutics, 10^{th} Ed., McGraw Hill, 2001 (referred to herein as Goodman and Gilman). In the event of a conflict or inconsistency between any of the incorporated references and the instant specification or the understanding of one or ordinary skill in the art, the specification shall control, it being understood that the determination of whether a conflict or inconsistency exists is within the discretion of the inventors and can be made at any time.

Where ranges of numerical values are stated herein, the endpoints are included within the range unless otherwise stated or otherwise evident from the context. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

This application refers to various genes and proteins using names that are well known in the art. At times one or more identifiers and/or accession numbers for these genes and proteins are provided. Such names, identifiers, and/or accession numbers are utilized in various databases available to one of skill in the art such as Genbank and Pubmed. For example, one of skill in the art can search the Entrez Gene database provided by the National Center for Biotechnology Information (NCBI), available at the web site having URL www.ncbi.nlm.nih.gov/entrez/query.fcgi?CMD=search&DB=gene and can thereby locate the Gene ID for any particular gene or protein of interest. The Gene ID entry provides biological information, alternate names, chromosomal location, etc., as well as links to database entries for the corresponding nucleotide and protein sequences and references in the scientific literature. Unless otherwise indicated, Gene IDs presented herein refer to the human form of the gene.

### BRIEF DESCRIPTION OF THE DRAWING

FIGS. 1A-1E present photographs and a bar graph showing that spine motility is elevated in vivo contralateral to the deprived eye following short monocular deprivation during the critical period. A, Apical dendritic arbors from mice expressing GFP in a subset of their layer V pyramidal neurons are visualized with two-photon microscopy. The top panel shows a collapsed z-stack of the arbor from a top-down view and the lower panel shows a side-view of the same arbor after a volumetric projection in the x-z axis. Scale bar, 50 μm in the x and y axes, 100 μm in the z axis. B, The location of in vivo imaging is marked with an injection of Alexa Fluor 594 which is then used to verify that the imaged neuron was in the binocular region of V1. Scale bar, 500 μm. C, The same neuron as in B at higher magnification showing the classic apical arbor of a layer V pyramidal neuron. Scale bar, 100 μm. D, A sample image (the first image of a time series) indicates that spines are readily identifiable in vivo with sufficient signal to noise that they can be tracked reliably over several hours. Scale bar, 5 μm. E, Spine motility is significantly elevated following brief deprivation during the critical period. Black bars, spines from non-deprived cortex, red bars, spines from cortex contralateral to the deprived eye. Double asterisk, p<0.0001, single asterisk, p<0.005.

FIGS. 2A-2E present photographs and bar graphs showing that spine motility is elevated in acute slice following short monocular deprivation. A, GFP expressing neurons are identified in the binocular portion of V1. Scale bar, 500 μm. B, The two neurons shown boxed in A are imaged at 60× with two-photon microscopy. Scale bar, 50 μm. C, A population of spines, proximal to the cell body, from the region shown in B are imaged every 6 minutes for 90 minutes. Scale bar, 10 μm. D, Spine motility is elevated following deprivation during the critical period in acute slice. E, There is no apparent change in average spine length, neck diameter, or head diameter. Black bars, spines from non-deprived cortex, red bars, spines from cortex contralateral to the deprived eye. Asterisk, p<0.0001.

FIGS. 3A-3D present photographs and bar charts showing that the upregulation of spine motility after monocular deprivation follows a laminar pattern. A, A large population of cells expressing GFP can be seen at 10× magnification, many of them extending long apical dendritic arbors. Scale bar, 300 μm. B, The three boxed regions shown in a at 60× magnification, progressing from regions close to the cell body (left panel), midway up the apical arbor (middle panel) and distal from the cell body (right panel). Below each 60× image is a high magnification image of the population of spines from the boxed region in the upper panels. Scale bars, 50 μm top panels, 10 μm for lower panels. C, If spines are grouped based on their distance from the cell body, a clear laminar pattern to the elevation of spine motility is observed. D, Similarly, if spines are grouped based on their distance from the cortical surface, the same laminar pattern is evident. Black bars, spines from non-deprived cortex, red bars, spines from cortex contralateral to the deprived eye. Asterisk, p<0.005.

FIGS. 4A and 4B present graphs and bar charts showing that spine motility is upregulated by degradation of the extracellular matrix. For each experimental condition, the blue and green traces in the left panels describe the change in length over time for two example spines before and after enzymatic degradation. Likewise, the middle panels show the motility index of a population of spines from a single experiment, including the spines from the left panel. The final column of panels shows the pooled effect of enzymatic degradation from all experiments. A, Spine motility is significantly elevated after proteolytic degradation with plasmin. B, Similarly, spine motility is significantly elevated after application of tPA with no exogenous plasminogen. Black bars, spines from non-deprived cortex, dark red bars, spines from non-deprived cortex following enzyme degradation. Asterisk, p<0.0001.

FIGS. 5A and 5B present graphs and bar charts showing that monocular deprivation occludes extracellular matrix degradation in a laminar fashion. The change in length of example spines, motility before and after extracellular matrix degradation, and pooled population results are as in FIG. 4. A, After monocular deprivation, plasmin significantly upregulates spine motility in the middle of the dendritic arbor. B, However, in the superficial part of the dendritic arbor, where spine motility is already upregulated by monocular deprivation, further upregulation by plasmin is occluded. Red bars, spines from cortex contralateral to the deprived eye, dark red bars, spines from cortex contralateral to the deprived eye following enzyme application. Asterisk, p<0.0001.

FIG. 6 is a schematic diagram showing the proposed time course of functional and structural changes following monocular deprivation and key elements of extracellular matrix remodeling. Monocular deprivation during the critical period induces tPA secretion and the conversion of extracellular plasminogen into plasmin. Plasmin then acts on a number of molecules in the extracellular matrix, allowing increased structural dynamics (schematically depicted with wavy lines around spines). This increase in structural dynamics then facilitates a change in synaptic connectivity, such that spines receiving input from the deprived eye (shown in red) are either lost or converted, while those spines receiving input from the open eye (shown in blue) are maintained.

FIGS. 7A and 7B show average tPA release from a hydrogel disc over a 14 day time course measured by ELISA (in IU/ml). (A) A small loading dose 2 μg is placed in the hydrogel. The control is a hydrogel disc made of the same material without any loaded tPA. Control levels were undetectable. (B) A larger loading dose of 33 μg is placed in each hydrogel. Double-stranded tPA refers to the two-chain form of tPA, which has a higher activity than the single-chain (referred to as single-stranded) form. The first day release concentrations are probably greater than the range of the ELISA, thus the amount of tPA released on day 1 is understimated in these figures.

FIGS. 8A and 8B show the effect of tPA on recovery of locomotor function graded on the BBB scale (y-axis; Basso et al., 1995 J. Neurotrauma) that ranges from 21 in normal rats to 0 in rats with complete hind limb paralysis. FIG. 8A presents a time course in which data points represent the group average±SEM (n=3/group) scored at different time points post T10 contusive injury. Results were analyzed with repeated measures ANOVA, which showed an overall significant (p<0.05) effect of treatment. Asterisks indicate that means are significantly different from the vehicle-treated control group at the specified times after SCI (Tukey's procedure). The dip of the curves starting at week 7 in both of the experimental groups was caused by the intra-cerebral cortex injections of axonal tract tracer BDA which was done at week 6 after injury. FIG. 8B presents a bar graph showing results of a second experiment in which data points represent the group average±SEM (n=7/group). Results were analyzed with repeated measures ANOVA, which showed an overall significant (p<0.05) effect of treatment. Asterisks indicate that means are significantly different from the vehicle-treated control group at the specified times after SCI (Tukey's procedure).

FIG. 9 presents micrographs showing the overall microscopic difference in injury epicenter pathology between rats receiving tPA or vehicle in the area of residual total tissue (hematoxylin and eosin stain). Sections were stained with hematoxylin and eosin. tPA treatment (left panel) produced a much cleaner parenchyma with more healthy tissue appearance versus that of vehicle treated rats (right panel) which showed diffuse tissue degeneration in the spared spinal cord residual tissue mass.

FIG. 10 presents micrographs showing the overall difference between rats receiving tPA (left panel) or vehicle (right panel) in the typical cross-sectional area of residual total white matter (WM) at the injury epicenter (e.g. WM+hypomyelinated WM). Sections were stained with solvent blue [SB]/hematoxylin and eosin as described in Teng and Wrathall, 1997. tPA treatment significantly protected the integrity of the residual white matter, showing high quality myelin stain in the spared white matter which demonstrates existence of myelinated axons. In contrast, SB stain in the vehicle treated spinal cord was much weaker, indicating much lower quantity presence of myelin in the tissue. The vehicle-treated epicenter also showed large scale infiltration of non neural tissue.

FIG. 11 shows a high magnification microscopic image of the spinal cord longitudinal profile of rats receiving tPA treatment in the typical area of residual total white matter (WM) at the injury epicenter (e.g. WM+hypomyelinated WM). Sections were stained with solvent blue [SB]/hematoxylin and eosin as described in Teng and Wrathall, 1997. High quality myelin stain is evident in the spared white matter which frequently showed longitudinally organized axonal arrays with healthy appearing myelin sheets (red arrows).

FIG. 12 presents micrographs showing BDA (biotinylated dextran amine) tracing of the corticospinal (CST) tract in a tPA treated animal. BDA was injected into the sensorimotor cortex at or beyond 42 days post injury such that BDA-positive fibers would be visible at rostral to, the site of, or caudal to, the spinal cord lesion if corticospinal tract fibers were present and passing through the injury site. Arrows indicate where the tracer passes into sites rostral to the epicenter (left panel) and the lesioned side of the cord (right panel). Note the tortuous path of the CST fibers (red arrows) that existed even within the lesioned zone of the parenchyma (right panel). Such regenerated or sprouted neurites may underlie as anatomical basis for the better preserved white matter quality demonstrated in FIG. 11, where SB stain depicted a much more healthy morphology of the white matter in terms of its myelinated axonal contents. (Scale bar: 80 μm in the left panel; 20 μm in the right panel).

### DETAILED DESCRIPTION OF CERTAIN EMBODIMENTS OF THE INVENTION

I. Definitions

The following definitions are of use in understanding the invention.

As used herein, the term “approximately” in reference to a number is generally taken to include numbers that fall within a range of 10% in either direction (greater than or less than) of the number unless otherwise stated or otherwise evident from the context (except where such number would exceed 100% of a possible value).

A material is considered “biocompatible” if it is substantially non-toxic to the recipient, in the quantities and at the location used, and also does not elicit or cause a significant deleterious or untoward effect on the recipient's body, e.g., a significant immunological or inflammatory reaction, unacceptable scar tissue formation, etc.

“Biodegradable”, as used herein, refers to a material that is capable of being broken down physically and/or chemically within the body of a subject, e.g., by hydrolysis under physiological conditions, by natural biological processes such as the action of enzymes present within the body, etc., to form smaller chemical species which can be metabolized and/or excreted.

The “central nervous system” (CNS) includes the brain, spinal cord, optic, olfactory, and auditory systems. The CNS comprises both neurons and glial cells (neuroglia), which are support cells that aid the function of neurons. Oligodendrocytes, astrocytes, and microglia are glial cells within the CNS. Oligodendrocytes myelinate axons in the CNS, while astrocytes contribute to the blood-brain barrier, which separates the CNS from blood proteins and cells, and perform a number of supportive functions for neurons. Microglial cells serve immune system functions.

“Concurrent administration”, as used herein with respect to two or more agents, e.g., therapeutic agents, is administration performed using doses and time intervals such that the administered agents are present together within the body, or at a site of action in the body such as in the CNS over a time interval in less than de minimis quantities. The time interval can be minutes, hours, days, weeks, etc. Accordingly, the agents may, but need not be, administered together as part of a single composition. In addition, the agents may, but need not be, administered simultaneously (e.g., within less than 5 minutes, or within less than 1 minute) or within a short time of one another (e.g., less than 1 hour, less than 30 minutes, less than 10 minutes, approximately 5 minutes apart). According to various embodiments of the invention agents administered within such time intervals may be considered to be administered at substantially the same time. One of ordinary skill in the art will be able to readily determine appropriate doses and time interval between administration of the agents so that they will each be present at more than de minimis levels within the body or, preferably, at effective concentrations within the body. When administered concurrently, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

An “effective amount” of an active agent refers to the amount of the active agent sufficient to elicit a desired biological response. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. A desired biological response may be, for example, (i) a reorganization of synaptic connections; (ii) a regeneration of a nerve or an axonal projection system; (iii) an improvement in performance of a task requiring motor function; (iv) an improvement in performance of a task requiring sensory function; (v) an improvement in performance of a task requiring cognitive function, e.g., improved performance on a test that measures learning and/or memory; (vi) a slowing in the rate of decline in motor, sensory, and/or cognitive function.

“Focal delivery”, or “focal administration” in reference to delivery of a pharmacological agent such as tPA or other agents mentioned herein, refers to delivery that does not rely upon transport of the agent to its intended target tissue via the vascular system, e.g., the agent is not administered directly into a blood vessel. The agent is delivered directly to its intended target tissue or in the vicinity thereof, e.g. by injection through a needle, catheter, or cannula, or by implantation of a delivery vehicle or device containing the agent. If the agent is delivered to the vicinity of its target tissue rather than into the target tissue itself, the agent may reach its target tissue by diffusion. For purposes of the present invention, any method that achieves delivery of an agent to the CNS or portion thereof without requiring transport via the vascular system from a site outside the skull or meninges (the membranes that cover the brain and the spinal cord), is considered to achieve focal delivery of the agent. Specifically included are delivery by use of an implanted or external pump, and/or delivery directly into one or more ventricles of the CNS. It will be understood that once having been focally delivered a portion of the agent (typically only a minor fraction thereof) may in part enter the vascular system and be transported to another location.

The term “function”, with reference to the nervous system or a component thereof, is used broadly herein to refer to any function, role, task, or activity performed by the nervous system or a component thereof. The term includes, without limitation, the ability to process and recall information, regulate behavior, stimulate release of endogenous chemicals, control motor functions, receive and process sensory input, maintain consciousness, etc.

“Functional recovery”, as used herein, refers to the process in which a nervous system or component thereof that has at least in part lost the ability to perform a function that it previously performed, regains at least in part the ability to perform the function. Functional recovery may take place in at least two different ways: (i) the recovery in function may involve partial or complete recovery of the portion or region of the nervous system that previously performed the function; (ii) the recovery in function may involve a portion or region of the nervous system performing a function that it did not previously perform. Of course in some instances both processes may take place. Functional recovery can also refer to preservation of the ability of the nervous system or a component thereof to perform a function that it previously performed, after the nervous system or component thereof has been physically altered, disrupted, or otherwise subjected to a physical or chemical insult or neurodegenerative disease, when such physical alteration, disruption, physical or chemical insult or neurodegenerative disease would otherwise be expected to lead to deterioration or loss of the ability of the nervous system or component thereof to perform the function.

“Functional reorganization”, as used in reference to the nervous system or a portion thereof, refers to the process in which a portion or region of the nervous system to wholly or partially assume, i.e., take on, a function (e.g., a sensory, motor, or cognitive function) that was not previously performed by that portion or region of the nervous system. The function or task may, but need not have been, previously performed by a different region or portion of the nervous system. The portion or region of the nervous system may be as small as a single neuron. Functional reorganization may, but need not, entail one or more aspects of structural reorganization. Functional reorganization may also be referred to as functional rearrangement.

An example of functional reorganization is the capacity of an area of sensory or motor cortex adjacent to an area of injury or necrosis of CNS tissue to control CNS output to a portion of the body that was previously controlled by the injured or necrotic tissue, or to receive and process input from a region of the body from which input was previously received and processed by the injured or necrotic tissue. Another example is the capacity of an area of sensory or motor cortex corresponding in location to an area of injury or necrosis of CNS tissue, but located in the opposite hemisphere of the brain, to control CNS output to a portion of the body that was previously controlled by the injured or necrotic tissue, or to receive and process input from a region of the body from which input was previously received and processed by the injured or necrotic tissue. Yet another example is provided by the nervous system's response to monocular deprivation, which is further discussed below.

An “infarct” is an area of localized tissue necrosis resulting from inadequate blood supply, e.g., due to obstruction of a blood vessel. Also referred to as an infarction. When the necrotic tissue is brain tissue, the infarct may be referred to as a cerebral infarct or cerebral infarction.

“Neural tissue”, for purposes of this invention refers to one or more components of the central nervous system and/or peripheral nervous system. Such components include brain tissue and nerves, which may be present in bundles or tracts. In general, brain tissue and nerves contain neurons (which typically comprise cell body, axon, and dendrite(s)), glial cells (e.g., astrocytes, oligodendrocytes, and microglia in the CNS; Schwann cells in the PNS). It will be appreciated that brain tissue and nerves typically also contain various noncellular supporting materials such as basal lamina (in the PNS), endoneurium, perineurium, and epineuriun in nerves, etc. Additional nonneural cells such as fibroblasts, endothelial cells, macrophages, etc., are typically also present. See Schmidt and Leach, 2003, for further description of the structure of various neural tissues.

The “peripheral nervous system” (PNS), for purposed of the present invention, includes the cranial nerves arising from the brain (other than the optic and olfactory nerves), the spinal nerves arising from the spinal cord, sensory nerve cell bodies, and their processes, i.e., all nervous tissue outside of the CNS. The PNS comprises both neurons and glial cells (neuroglia), which are support cells that aid the function of neurons. Glial cells within the PNS are known as Schwann cells, and serve to myelinate axons by providing a sheath that surrounds the axons. In various embodiments of the invention the methods and compositions described herein are applied to different portions of the PNS.

“Plasticity” refers to the capacity of the nervous system, or a portion thereof, to change (e.g., to reorganize) its structure and/or function, generally in response to an environmental condition, injury, experience, or ongoing nervous system activity. Plasticity may involve the proliferation of neurons or glia, the growth or movement of neuronal processes and/or alterations in their shape. Plasticity may involve formation of new synaptic connections between neurons and/or strengthening or weakening of existing synaptic connections. Formation of new synaptic connections may involve growth or movement of neuronal processes. Plasticity may also involve alterations in non-neuronal components of the nervous system, e.g., astrocytes or other glial cells.

A “polypeptide” is a polymer of amino acids. A “protein” is a molecule composed of one or more polypeptides. The terms “protein”, “polypeptide”, and “peptide” may be used interchangeably herein. Polypeptides as described herein typically contain only natural amino acids, although non-natural amino acids (i.e., compounds that do not occur in nature in polypeptides but that can be incorporated into a polypeptide chain) and/or amino acid analogs as are known in the art may also be employed.

“Proteolysis” refers to the breakdown, or degradation, of proteins into smaller polypeptides, typically by cleavage of peptide bonds. Ultimately proteolysis may result in breakdown of the protein into individual amino acids.

“Proteolysis-enhancing agent”, as used herein, is an agent, e.g., a protease, that increases, contributes to, or causes proteolysis of one or more proteins or inhibits an inhibitor of proteolysis.

“Purified”, as used herein, means separated from many other compounds or entities. A compound or entity may be partially purified, substantially purified, or pure, where it is pure when it is removed from substantially all other compounds or entities (other than solvents, ions, etc.), i.e., it is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids. In a preferred embodiment a purified protein is removed from at least 90%, preferably at least 95%, more preferably at least 99%, or more, of the other proteins in a preparation, so that the purified protein constitutes at least 90%, preferably at least 95%, more preferably at least 99%, of the material in the preparation on a dry w/w basis.

“Sequential administration” of two or more agents refers to administration of two or more agents to a subject such that the agents are not present together in the subject's body at greater than de minimis concentrations. Administration of the agents may, but need not, alternate. Each agent may be administered multiple times.

“Small molecule”, as used herein, refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

“Spine dynamics” refers to a change in any of various structural properties of spines over time. The properties include spine shape, size, number, density, and motility. Spine dynamics may be examined with respect to the individual spine or with respect to a plurality (i.e, more than one) of spines.

“Spine motility” refers to a change in spine length over time. When examined with respect to a plurality of spines, spine motility refers to the average change in spine length over time.

“Structural recovery”, as used in reference to the nervous system or a portion thereof, refers to the partial or complete restoration of a structure that has physically altered, disrupted, or otherwise subjected to a physical or chemical insult, which is intended to include deprivation of oxygen and/or nutrients. “Structural recovery” can also refer to preservation of a structure that has been physically altered, disrupted, or otherwise subjected to a physical or chemical insult, when such physical alteration, disruption, physical or chemical insult would otherwise be expected to lead to deterioration and/or loss or alteration in normal structural features. The structure can be, for example, a synaptic connection, a nerve, nerve bundle, nerve tract, nucleus, brain region, connection between brain regions, etc.

“Structural reorganization”, as used in reference to the nervous system or a portion thereof, refers to an alteration in the pattern of connections between two or more neurons or between one or more neurons and one or more glial cells (e.g., astrocytes, oligodendrocytes, microglia, Schwann cells) that takes place over a period of time or an alteration in the position of two or more neuronal or glial cell bodies or cell processes (axons, dendrites, dendritic spines) with respect to one another. The alteration may include the formation of synapses between neurons that did not synapse with each other at the beginning of the time period. The alteration may include the formation of additional synapses between neurons that had at least one synaptic connection at the beginning of the time period. The alteration may also or alternatively include loss of synapses that existed at the beginning of the time period. Reorganization may entail growth or retraction of neural processes such as axons (e.g., axonal sprouting or regeneration), dendrites, or dendritic spines, migration of neurons or glia, and/or neuronal or glial cell division. Structural reorganization may also be referred to as structural rearrangement.

As used herein, “subject” refers to an individual to whom an agent is to be delivered, e.g., for experimental, diagnostic, and/or therapeutic purposes. Preferred subjects are mammals, particularly domesticated mammals (e.g., dogs, cats, etc.), primates, or humans.

“Synapses” are “specialized intercellularjunctions between neurons or between neurons and other excitable cells where signals are propagated from one cell to another with high spatial precision and speed.” (De Camilli, in Cowan, supra). They are the primary sites of intercellular communication in the mammalian nervous system. In general, the basic structure of a synapse consists of a close juxtaposition of specialized regions of the plasma membrane of two neurons, referred to as the presynaptic and postsynaptic neurons, to form a synaptic junction. The presynaptic neuron is the nerve cell transmitting a signal while the postsynaptic neuron is the recipient of the signal. Most neurons in the vertebrate nervous system possess a cell body and two types of cell processes, axons and dendrites. Signals, i.e., action potentials, are initiated and transmitted by the axon while dendrites (and also the cell body) receive inputs via synaptic contacts from other neurons.

“Treating”, generally refers to medical and/or surgical management of a patient for purposes of bringing about an improvement in the state of a subject with respect to a disease, disorder, or condition from which the subject suffers and/or reducing or slowing further deterioration of the subject's condition. Treating can include reversing, alleviating, and/or inhibiting the progress of, the disease, disorder, or condition to which such term applies, and/or reversing, alleviating, inhibiting the progress one or more symptoms or manifestations of such disease, disorder or condition.

II. Compositions and Methods for Promoting Nervous System Reorganization and Recovery

Conditions and events such as infarcts, traumatic brain injury, or spinal cord lesions typically result in extensive death of CNS tissue and a consequent loss of cellular connections (e.g., synaptic connections), often leading to long-term debilitation. However, it has been observed that the CNS exhibits significant capacity for recovery even in situations in which irreversible loss of CNS tissue has occurred, and functional deficits that are present immediately or shortly after the initial insult can improve over time. Such improvement may be accompanied by considerable functional reorganization (Johannsen 2000). For example, sensory or motor recovery following a stroke may be associated with adjacent or contralateral areas of cortex taking over the function of the damaged areas and/or the utilization of alternative sensory or motor pathways (Chen, R., et al., 2002; Nelles, G., et al., 1999; Cramer, S., et al., 1997; Xerri, C. et al., 1998). Unfortunately, although the CNS does appear to have the capacity to undergo reorganization following damage, such reorganization often fails to restore meaningful levels of function, and complete recovery of function is even more unusual, particularly after severe damage.

The present invention relates to methods and compositions for enhancing nervous system reorganization and/or recovery following damage to the central or peripheral nervous system of an individual, also referred to herein as a “subject”. While not wishing to be bound by any theory, the agents described herein may help to create an environment that is more permissive for structural and/or functional reorganization of neural tissue than would otherwise be the case. For example, the agents may cause degradation of molecules present in the ECM that would otherwise impede beneficial structural changes or would exert inhibitory effects on nervous system cells. Functional recovery from damaging events may involve regrowth of physical connections (e.g., synapses) between surviving nervous system cells (neurons, glia) and/or establishment of new connections. The compositions may at least in part cause degradation of one or more constituents of the so-called glial scar, which consists largely of astrocytes and extracellular matrix (ECM) components and may form a physical barrier to axonal growth (Fawcett and Asher, 1999). The agents may alternatively or additionally interact directly with cells, e.g., neurons, glial cells, etc., to stimulate their capacity for structural and/or functional reorganization.

The description herein focuses largely on the CNS, but the compositions and methods may also be applied to enhance reorganization and/or recovery following damage to the PNS. The compositions may be introduced into either the CNS or PNS for purposes of enhancing functional recovery of the PNS. It has been shown that regions of the CNS undergo functional reorganization after damage to the PNS. Peripheral injury leads to reorganization of both the sensory and motor systems at multiple levels including spinal cord, brainstem, thalamus and cortex (Chen et al., 2002). The compositions may also or alternatively be introduced into the PNS, e.g., at or in the vicinity of a site of injury to a peripheral nerve.

The present invention arose in part as a result of experiments performed by the inventors in the context of a widely used model for nervous system plasticity, namely monocular deprivation (MD). In animals with binocular vision, inputs to a portion of the visual cortex become anatomically and functionally segregated into alternating stripes of input from the two eyes, referred to as ocular dominance columns. As a consequence, individual cortical neurons that were originally responsive to both eyes become responsive to only one eye. However, if one eye is deprived of visual input during a critical period (monocular deprivation), that eye loses most of its ability to activate the cortex, and the responses of cells shift towards the nondeprived eye eye, i.e., ocular dominance (OD) shifts in favor of the nondeprived eye. The rapid appearance of the functional deficit is followed by structural changes including a reduction in cortical area driven by the deprived eye and expansion of the area driven by the nondeprived eye, which take place on a timescale of weeks to months. The extent and complexity of thalamocortical axonal arbors from the deprived eye are reduced, while the extent and complexity of arbors from the nondeprived eye increase. This indicates that following MD, processes at the level of synapses may guide large-scale reorganization of anatomical connectivity.

As described in more detail in Examples 1 and 2, the inventors examined rapid structural changes at the level of synapses by imaging dendritic spines both in vitro and in vivo following brief monocular deprivation. They found that such deprivation initiates a rapid upregulation in spine dynamics, demonstrating that alterations in spine dynamics are closely linked to functional alterations and likely to play a role in long-term functional and structural reorganization of the nervous system.

Briefly, dendritic spines are structural specializations that protrude from the shaft of dendrites and contain the post-synaptic elements of excitatory synapses (Hering and Sheng, 2001). Spines receive the majority of excitatory input in the mammalian CNS (Gray, 1959), and a typical mature spine has a single synapse located at its head. Spines have been morphologically classified into several groups on the basis of shape. However, spines are motile and dynamic structures, exhibiting changes in size and shape over time (Dunaevsky et al., 1999; Fischer et al., 1998). In addition to morphological changes that occur over hours or days, spines also show a much more rapid motility, exhibiting an ability to change shape on a timescale of seconds to minutes. These changes involve remodelling of the actin-based cytoskeleton (Fischer et al., 1998; Star et al., 2002).

Spine dynamics are likely to play a role in the functional properties of spines and thus of the dendrites from which they protrude, ultimately influencing the signal received by the neuron. Considerable evidence suggests that spine motility plays an important role in synaptogenesis, i.e., the development of new synapses (Bonhoeffer 2002 and references therein). Spine dynamics decrease over development (Lendvai et al., 2000) and, by the critical period for ocular dominance plasticity in mice, spines have achieved a relatively stable state (Majewska and Sur, 2003). Spine formation and density can be affected by nervous system activity over short and long timescales. For example, binocular deprivation, by reducing visual cortical activity over long periods of time, increases spine motility (Majewska and Sur, 2003). This increase in dynamics may reflect a process by which spines are destabilized and actively try to increase their synaptic drive.

Given the likely importance of spine dynamics in structural and functional reorganization of the nervous system, the inventors sought to determine spine dynamics could be influenced by treatment with various agents. As described in Example 3, it was discovered that spine motility in an in vitro nervous system tissue preparation is significantly elevated following application of proteolysis-enhancing agents, in particular plasmin and tissue plasminogen activator (tPA). Furthermore, as described in Example 6, focal administration of tPA to animals who had been subjected to spinal cord injury caused significant improvement in and/or preservation of the structural features of the spinal cord tissue and significantly enhanced functional recovery relative to control animals who did not receive tPA. In addition, as described in Example 7, focal administration of tPA to animals who had been subjected to middle cerebral artery occlusion, causing infarction, exhibited improved responses on a behavioral test than control animals who did not receive tPA.

In one aspect, the invention provides a method for promoting reorganization or recovery in the nervous system of a subject comprising the step of: focally administering a composition comprising a proteolysis-enhancing agent to the central or peripheral nervous system of a subject in need of enhancement of reorganization or recovery of the nervous system as a result of ischemic, hemorrhagic, neoplastic, traumatic, neurodegenerative, or toxic damage to the nervous system. The agent contributes to, e.g., enhances, recovery or reorganization in the subject's nervous system. In other words, the degree of reorganization or recovery of the nervous system is greater than would have been the case if the agent had not been administered to the subject. By “reorganization” is meant structural and/or functional reorganization. By “recovery” is meant structural and/or functional recovery.

Typically the agents of the invention promote structural reorganization and/or functional reorganization of the nervous system or a portion thereof. Preferably the agents of the invention promote structural and/or functional recovery of the nervous system or a portion thereof. It will be appreciated that often there will be a correlation between (i) structural reorganization and/or recovery and (ii) functional reorganization and/or recovery, e.g., both structural reorganization and/or recovery as well as functional reorganization and/or recovery take place. However, in some embodiments of the invention functional reorganization and/or recovery take place without detectable evidence of structural reorganization and/or recovery. In some embodiments of the invention structural reorganization and/or recovery take place without detectable evidence of functional reorganization and/or recovery during a particular time period of evaluation. In such embodiments, functional reorganization and/or recovery may occur at a later time, and/or the recovery may not be detected using the particular measurement tools and methods used for the evaluation. It will also be appreciated that reorganization is typically associated with recovery, but reorganization can precede noticeable evidence of recovery, sometimes by a significant period of time.

To the best of the inventors' knowledge, direct evidence that treatment with components of the tPA/plasmin cascade can cause structural changes in a nervous system preparation that retains the structural features, architecture, and cell-cell and cell-ECM contacts of the intact nervous system has not previously been reported. Furthermore, to the best of the inventors' knowledge, the focal administration of proteolysis-enhancing agents, e.g., components of the tPA/plasmin cascade, to the nervous system to enhance structural and/or functional reorganization and recovery following damage to the nervous system has not previously been proposed. Without wishing to be bound by any theory, the inventors propose that proteolysis of one or more ECM component(s), mediated by tPA and/or plasmin, creates an environment that is permissive for structural reorganization. Accordingly, enhancing proteolytic activity in the nervous system following nervous system damage will permit increased structural remodeling, thereby contributing to improved functional recovery. The following sections describe proteolysis-enhancing agents of use in the invention, drug delivery devices, methods and locations for the focal administration of proteolysis-enhancing agents, and various other features of the invention.

III. Proteolysis-Enhancing Agents

A variety of different proteolysis-enhancing agents, or combinations thereof, are of use in the present invention. In certain embodiments of the invention the proteolysis-enhancing agent is a polypeptide. In certain embodiments of the invention the polypeptide is a protease. In certain embodiments of the invention the proteolysis-enhancing agent enhances proteolysis of fibrin. The agent may directly cleave fibrin or may activate an endogenous protease that cleaves fibrin. In certain embodiments of the invention the agent enhances proteolysis of a component of the ECM other than fibrin in addition to, or instead of, enhancig proteolysis of fibrin. For example, the proteolysis-enhancing agent may cleave one or more extracellular matrix components including, but not limited to, collagen, laminin, fibronectin and proteoglycans.

Suitable agents for use in the present invention include components of the tPA/plasmin cascade. Components of the tPA/plasmin cascade include plasminogen activators such as tissue plasminogen activator (tPA) and variants thereof, plasminogen, and plasmin. Plasminogen activators (PAs) are serine proteases that catalyze the conversion of plasminogen to plasmin (Vassalli 1991) by cleavage of a single peptide bond (R561-V562) yielding two chains that remain connected by two disulfide bridges (Higgins and Bennett, 1990). Plasmin is a potent serine protease whose major substrate in vivo is fibrin, the proteinaceous component of blood clots. Plasminogen activation by tPA is stimulated in the presence of fibrin. Plasmin has a broad substrate range and is capable of either directly or indirectly cleaving many other proteins, including most proteins found in the ECM. “Direct”, as used here, means that the protease physically interacts with the polypeptide that is cleaved, while “indirect” means that the protease does not physically interact with the polypeptide that is cleaved—instead it interacts with another molecule, e.g., another protease, which in turn directly or indirectly cleaves the polypeptide. Plasmin is also capable of activating metalloprotease precursors. The metalloproteases in turn degrade ECM molecules. Metalloproteases are also of use in certain embodiments of the present invention. In addition to the afore-mentioned substrates, plasmin cleaves and activates various growth factors and growth factor precursors. Although the liver is the major site of plasmin synthesis, plasminogen mRNA and protein have been detected in numerous brain regions. Thus plasminogen is available to be cleaved by tPA administered to the nervous system.

Two PAs, tissue-type PA (tPA) and urokinase-type PA (uPA) have been identified in mammals. A major physiological function of PAs to trigger the lysis of clots by activating plasminogen to plasmin, which degrades fibrin. In the body, PA activity is regulated in part by various endogenous serine protease inhibitors that inhibit PAs, a number of which have been identified. Neuroserpin (Gene ID 5274) belongs to the serpin family of the serine protease inhibitors and is expressed by neurons of both the developing and the adult nervous system. Neuroserpin is present in regions of the brain where either tPA message or tPA protein are also found, suggesting that neuroserpin is the selective inhibitor of tPA in the CNS. Plasminogen activator inhibitor 1 (PAI-1; Gene ID 5054) is the main plasminogen activator inhibitor (PAI) in plasma but is also found in the nervous system. Protease-nexin I (Gene ID 5270), PAI-2 (Gene ID 5055), and PAI-3 (Gene ID 268591, Mus musculus) are other endogenous PAIs. Protease-nexin I and neuroserpin inhibit plasmin in addition to PAs.

While not wishing to be bound by any theory, there are a number of potential substrates for tPA and/or plasmin whose proteolysis may contribute to structural reorganization in the nervous system. Among these are various ECM proteins such as fibrin, fibronectin, tenascin, and laminin. In addition to plasmin, tPA may activate other proteases such as the plasmin-like protein hepatocyte growth factor (HGF), which may in turn cleave additional substrates.

tPA for use in the present invention may be from any species, although for administration to humans it is generally preferred to use human tPA or a variant thereof. tPA and useful variants thereof, including variants with improved properties are described in U.S. Pat. Nos. 6,284,247; 6,261,837; 5,869,314; 5,770,426; 5,753,486 5,728,566; 5,728,565; 5,714,372; 5,616,486; 5,612,029; 5,587,159; 5,520,913; 5,520,911; 5,411,871; 5,385,732; 5,262,170; 5,185,259; 5,108,901; 4,766,075; 4,853,330, and other patents assigned to Genentech, Inc. See also Higgins 1990. For example, and without limitation, the tPA variant may have an alteration in the protease domain, relative to naturally occurring tPA, and/or may have a deletion of one or more amino acids at the N-terminus, relative to naturally occurring tPA. The tPA variant may have one or more additional glycosylation sites relative to naturally occurring tPA and/or may have an alteration that disrupts glycosylation that would normally occur in naturally occurring tPA when expressed in eukaryotic cells, e.g., mammalian cells. Properties that may be of use include, but are not limited to, increased half-life, increased activity, increased affinity or specificity for fibrin, etc.

Human tPA has been assigned Gene ID 5327 in the Entrez Gene database (National Center for Biotechnology Information; NCBI) and the GenBank entry for the full length amino acid, mRNA, and gene sequences are AAA98809, K03021, and NM_{—}000930, respectively. However, it is noted that it may be preferable to use the mature form of tPA, lacking the signal sequence peptide, as described, e.g., in U.S. Pat. No. 4,853,330 and Yelverton 1983, or a variant thereof.

The chymotrypsin family serine proteases, of which tPA is a member, are normally secreted as single chain proteins and are activated by a proteolytic cleavage at a specific site in the polypeptide chain to produce a two chain form (Renatus, 1997, and references therein). Both the single chain and two chain forms are active towards plasminogen, although the activity of the two-chain form is greater. Plasmin activates single-chain tPA to the two-chain form, thus resulting in a positive feedback loop. Either the single chain or the two chain form of tPA, or combinations thereof, may be used in the present invention.

tPA and variants thereof are commercially available and have been approved for administration to humans for a variety of conditions. For example alteplase (Activase®, Genentech, South San Franciso, Calif.) is recombinant human tPA. Reteplase (Retavase®, Rapilysin®; Boehringer Mannheim, Roche Centoror) is a recombinant non-glycosylated form of human tPA in which the molecule has been genetically engineered to contain 355 of the 527 amino acids of the original protein. Tenecteplase (TNKase®, Genentech) is a 527 amino acid glycoprotein derivative of human tPA that differs from naturally occurring human tPA by having three amino acid substitutions. These substitutions decrease plasma clearance, increase fibrin binding (and thereby increase fibrin specificity), and increase resistance to plasminogen activator inhibitor-1 (PAI-1). Anistreplase (Eminase®, SmithKline Beecham) is yet another commercially available human tPA.

Alternate plasminogen activators include streptokinase (Streptase®, Kabikinase®) and urokinase (Abbokinase®), both of which are commercially available.

Other proteolysis-enhancing agents of use in the invention include tPA activators such as Desmodus rotundus salivary plasminogen activator (DSPA) Desmoteplase (Paion, Germany) which is derived from vampire bat saliva (Liberatore, 2003, and references therein). Four distinct proteases have been characterized and are referred to as D rotundus salivary plasminogen activators (DSPAs). Full-length vampire bat plasminogen activator (DSPAI) is the variant most intensively studied and exhibits >72% amino acid sequence identity with human tPA. However, 2 important functional differences are apparent. First the DSPAs exist as single-chain molecules that are not cleaved into 2 chain forms. Second, the catalytic activity of the DSPAs appears to be dependent on a fibrin cofactor. Urokinase plasminogen activators such as rescupase (Saruplase®, Grunenthal), and microplasmin (a cleavage product of plasminogen) are also of use in various embodiments of the invention. Alfimeprase (Nuvelo) is yet another proteolysis-enhancing agent of use in the present invention. Alfimeprase is a recombinantly produced, truncated form of fibrolase, a known directly fibrinolytic zinc metalloproteinase that was first isolated from the venom of the southern copperhead snake (Agkistrodon contortrix contortrix) (Toombs, 2001). These enzymes breaks down fibrin directly. Fibrolase itself is also of use in the present invention. Also of use is staphylokinase (Schlott, 1997).

In some embodiments of the invention plasmin or mini-plasmin is administered instead of, or in addition to, tPA. A variety of other agents that have plasmin-like activity may also be used. In general, such substances are able to cleave typical plasmin substrates, such as the synthetic substrate S-2251™ (Chromogenix-Instrumentation Laboratory, Milan, Italy), which is a conveniently assayed chromogenic substrate for plasmin and activated plasminogen. Other agents that have tPA-like activity, e.g., they are able to cleave plasminogen and activate it in a similar manner to tPA, can also be used.

Lumbrokinase is an enzyme or group of enzymes derived from earthworms Lumbricus rubellus which has been known for some time. See, e.g., reporting cloning of a gene encoding lumbrokinase (PI239, GenBank Accession No. AF433650) (Ge, 2005). Other fibrinolytic proteases isolated from earthworms are also of use (Cho, 2004). Also of use is nattokinase.

Also of use are a variety of fibrinolytic enzymes that have been isolated from various worms, insects, and parasites. For example, destabilase, an enzyme present in the leech, hydrolyzes fibrin cross-links (Zavalova, 1996). See also Zavalova, 2002.

In some embodiments of the invention plasminogen is administered instead of, or in addition to, tPA.

Instead of, or in addition to, administering a molecule that itself has plasminogen activator activity, plasmin activity, or plasmin-like activity, substances that increase endogenous expression of plasminogen activators or plasmin may be administered. Such substances may act by increasing transcription or translation of the mRNA that encodes the molecule, stabilizes the molecule, etc. They include, but are not limited to, brain derived neurotrophic factor (BDNF), transforming growth factor-β (TGF-β), phorbol esters, and retinoic acid.

A variety of other agents can be administered to enhance protolysis in the central or peripheral nervous system in order to treat nervous system damage due to ischemic, hemorrhagic, neoplastic, traumatic, or degenerative conditions. Certain of these agents are administered focally while others are administered using an alternate route of admistration, e.g., oral, intravenous, intramuscular, subcutaneous, pulmonary, nasal, etc. For example, sulodexide is a fibrinolytic agent that releases cellular tPA and thus is of use to increase tPA activity. In certain embodiments of the invention it is administered orally (Harenberg, 1998). Other agents of use in the invention to inhibit PAI include enalapril (Sakata, 1999) and ampotherin (Parkinnen, 1993).

Aspirin, which has been reported to stimulate plasmin activity, is also of use in the invention (Milwidksy, 1991). In certain embodiments aspirin is not used, or if the subject is receiving aspirin, a different agent is used in addition to aspirin.

Another strategy that may be used to increase the level of plasminogen activator activity, plasmin activity, or plasmin-like activity is to administer a substance that inhibits one or more of the endogenous inhibitors of tPA or plasmin. Such endogenous inhibitors include PAI-1, PAI-2, PAI-3, and neuroserpin. A plasminogen activator inhibitor will be referred to as a PAI herein. In one embodiment, an inactive form of a PAI, which is unable to inhibit plasminogen activators, is used. See, e.g., WO 97/39028 and Lawrence et al., J Biol. Chem. 272:7676-7680 (1997) which describe various inactive forms of PAI. Without wishing to be bound by any theory, an inactive form of PAI may compete with an active form and thereby prevent inhibition of tPA. Small molecules and peptides that inhibit one or more PA1s are known in the art and are of use in the present invention. Examples include PAI-039 (Hennan, 2005), ZK4044 (Liang, 2005), tiplaxtinin (Elokdah, 2004), piperazine-based derivatives (Ye, 2004), T-686 (Ohtani, 1996), fendosal (HP129), AR-H029953XX, XR1853, XR5118 and the peptide TVASS (Gils, 2002).

RNA-mediated interference (RNAi) has recently emerged as a powerful method to reduce the expression of any target transcript in mammalian cells (See, e.g., Elbashir, 2001; Brummelkamp, 2002; McManus & Sharp, 2002; U.S. Pub. Nos. 20050026278; 20040259248; and 20030108923) Briefly, it has been found that the presence within a cell of a short double-stranded RNA molecule referred to as a short interfering RNA (siRNA), one strand of which is substantially complementary to a transcript present in the cell (the target transcript) over a length of about 17-29 nucleotides, results in inhibition of expression of the target transcript. The mechanism typically involves degradation of the transcript by intracellar machinery that cleaves RNA (although translational inhibition can also occur). Short hairpin RNAs are single-stranded RNA molecules that include a stem (formed by self-hybridization of two complementary portions of the RNA) and a loop, which can be processed intracellularly into siRNA.

RNAi may be used to reduce expression of a transcript that encodes an inhibitory protein, e.g., an endogenous PAI. siRNAs or shRNAs targeted to a transcript that encodes an inhibitory protein can be delivered together with a proteolysis-enhancing agent or administered separately. Alternatively, a vector that provides a template for intracellular synthesis of one or more RNAs that hybridize to each other or self-hybridize to form an siRNA or shRNA that inhibits expression of an inhibitory protein, or cells that synthesize such RNAs, can be administered.

Antisense oligonucleotides complementary to an mRNA transcript that encodes an inhibitory protein, or ribozymes that cleave the transcript, can also be used to downregulate expression of the inhibitor. Antisense oligonucleotides can be administered together with a proteolysis-enhancing agent or separately. Alternately, a vector that provides a template for intracellular synthesis of an antisense oligonucleotide, or cells that synthesize the oligonucleotide, can be administered. Antisense technology and its applications are well known in the art and are described in Phillips, M. I. (ed.) Antisense Technology, Methods Enzymol., Volumes 313 and 314, Academic Press, San Diego, 2000, and references mentioned therein. See also Crooke, S. (ed.) “Antisense Drug Technology: Principles, Strategies, and Applications” (1st ed), Marcel Dekker; ISBN: 0824705661; 1st edition (2001) and references therein.

In another embodiment of the invention an aptamer that binds to a PAI and inhibits its inhibitory activity is used. An aptamer is an oligonucleotide (e.g., DNA, RNA, which can include various modified nucleotides, e.g., 2′-O-methyl modified nucleotides) that binds to a particular protein. Aptamers are typically derived from an in vitro evolution process (SELEX), and methods for obtaining aptamers specific for a protein of interest are known in the art. See, e.g., Brody, 2000.

RNA and DNA molecules can act as enzymes by folding into a catalytically active structure that is specified by the nucleotide sequence of the molecule. Certain of these molecules are referred to as ribozymes or deoxyribozymes. In particular, both RNA and DNA molecules have been shown to catalyze the sequence-specific cleavage of RNA molecules. The cleavage site is determined by complementary pairing of nucleotides in the RNA or DNA enzyme with nucleotides in the target RNA. Thus, RNA and DNA enzymes can be designed to cleave to any RNA molecule, thereby increasing its rate of degradation (Cotten and Birnstiel, 1989; Usman, 1996; Sun, 2000). In another embodiment of the invention an RNA or DNA enzyme that cleaves a transcript that encodes a PAI and thus inhibits its inhibitory activity is used.

It will be appreciated that nucleic acids such as siRNA, antisense oligonucleotides, aptamers, ribozymes, etc., can include various modifications to the nucleoside(s) and/or phosphodiester backbone.

In other embodiments an antibody or antibody fragment that binds to a PAI is used to inhibit its activity, or any polypeptide having a similar binding specificity, e.g., an affibody. The antibody or antibody fragment can be any immunoglobulin or immunoglobulin-like molecule that binds to an antigen and can be monoclonal or polyclonal.

Any substance that acts to counteract the effect of a molecule that is inhibitory for activity of a proteolysis-enhancing agent, whether by causing degradation, by sequestering, by reducing expression, or by blocking interaction of the molecule with another molecule or with a cell will be said to counteract the inhibitory molecule and is within the scope and spirit of the invention.

As described above, the inventors hypothesize that enhancing proteolytic activity in the nervous system following nervous system damage will permit increased structural remodeling, thereby contributing to improved functional recovery. However, the invention described herein does not require any particular mechanism of action. The agents described herein need not necessarily exert their effects on nervous system reorganization and/or recovery by enhancing proteolysis. In certain embodiments of the invention the agents disclosed herein, e.g., tPA and variants thereof, plasmin, etc., do not in fact enhance proteolysis. For example, the invention encompasses variants of these proteases (e.g., variants having a mutation in an active site region) in which the sequence has been altered, such that the variant is no longer an active proteolytic agent. The invention also encompasses embodiments in which the proteolysis-enhancing agent has been chemically inactivated, such that it no longer enhances proteolysis. Thus in some embodiments of the invention an inactive form of a proteolysis-enhancing agent is focally administered. However, in general, a proteolysis-enhancing agent is active or capable of being activated when used according to the present invention.

It will be appreciated that most proteins can tolerate a certain amount of sequence variation without substantial loss of functional activity, provided that such sequence variation does not affect key residues that are required for such functional activity. The present invention therefore encompasses variants of the proteolysis-enhancing polypeptides (and other polypeptides disclosed herein), wherein such variants retain a significant amount of biological activity. For example, the fragment can have substantially similar activity (e.g., at least about 10-20% of the relevant activity) to the original polypeptide, at least about 50% of the relevant activity, etc. The term “variants” includes fragments, i.e., polypeptides whose sequence is a continuous subset of a polypeptide disclosed herein. Specifically encompassed are variants or fragments in which one or more kringle domains of a polypeptide disclosed herein, e.g., plasmin or tPA, is removed. Certain fragments of use in this invention contain a protease domain and, optionally, at least one kringle domain.

As is well known in the art, certain amino acids are generally similar with respect to particular properties and can frequently be substituted for one another in a polypeptide without significantly altering the functional and structural properties of the polypeptide. For example, the variants may contain one or more conservative amino acid substitutions, which may be defined in accordance with Stryer, L. Biochemistry, 3rd ed., 1988. Amino acids in the following groups possess similar features with respect to side chain properties such as charge, hydrophobicity, aromaticity, etc., and can be substituted for one another in accordance with certain embodiments of the invention: (1) Aliphatic side chains: G, A, V, L, I; (2) Aromatic side chains: F, Y, W; (3) Sulfur-containing side chains: C, M; (4) Aliphatic hydroxyl side chains: S, T; (5) Basic side chains: K, R, H; (6) Acidic amino acids: D, E, N, Q; (7) Cyclic aliphatic side chain: P (P may be considered to fall within group (1)). One of ordinary skill in the art will recognize that other narrower definitions of conservative substitutions can also be used. Amino acid abbreviations used herein are in accordance with common usage in the art.

The present invention encompasses administration of variants that are at least 80% identical, preferably at least 90% identical, more preferably at least 95% identical to one or more of the polypeptides disclosed herein. Percent identity may be calculated by standard methods. For example, the percent identity between first and second nucleic acids over a window of evaluation may be computed by aligning the nucleic acids, determining the number of nucleotides within the window of evaluation that are opposite an identical nucleotide allowing the introduction of gaps to maximize identity, dividing by the total number of nucleotide positions in the window, and multiplying by 100. Various computer programs such as BLAST2, BLASTP, Gapped BLAST, etc., generate alignments and provide % identity between sequences of interest. Algorithms employed in those programs (utilizing default values) can be used.

The present invention encompasses variants in which up to 20%, or preferably up to 10%, or up to 5% of the amino acid residues are either substituted (e.g., conservatively substituted), deleted, or added, relative to a polypeptide disclosed herein. Specifically encompassed are allelic variants that exist within a population. The invention encompasses variants that are specifically recognized by immunological reagents (e.g., monoclonal or polyclonal antibodies) that recognize a polypeptide disclosed herein, i.e., the immunological reagent binds to the variant with a substantially similar affinity (e.g., having a Ka at least 50% as great) as that with which it binds to the polypeptide.

The invention encompasses variants that have a substantially similar overall structure to the polypeptides disclosed herein. For example, certain variants possess sufficient structural similarity to a protein disclosed herein so that when its 3-dimensional structure (either actual or predicted structure) is superimposed on the structure of the protein the volume of overlap is at least 70%, preferably at least 80%, more preferably at least 90% of the total volume of the structure. Structures of plasmin and tPA, optionally bound to substrate, are known in the art. Furthermore a partial or complete 3-dimensional structure of a variant may be determined by crystallizing the protein using methods known in the art. Alternately, an NMR solution structure can be generated. See, e.g., Heinemann 2001, Wishart D. 2005, and references therein). A modelling program such as MODELLER (Sali and Blundell, 1993), or any other modelling program, can be used to generate a predicted structure. The PROSPECT-PSPP suite of programs can be used (Guo, 2004).

In certain embodiments of the invention the variant also has substantially similar proteolysis-enhancing activity as the polypeptide of which it is a variant. In certain embodiments of the invention the variant does not have a substitution at an active site residue. Active site residues of serine proteases such as the proteases disclosed herein are well known in the art.

It will be appreciated that various agents have been focally administered to the nervous system of a subject suffering from ischemic, hemorrhagic, neoplastic, traumatic, toxic, and/or neurodegenerative damage to the nervous system, for purposes other than enhancing proteolysis. For example, analgesic agents are commonly administered. Should it be the case that any of such previously administered agents should happen to enhance proteolysis, such agent may be explicitly excluded from the present invention or, if used in the present invention, its use in the context of the present invention differs from such previous use. For example, its use in the context of the present invention involves administration to a different location, uses a different administration means, and/or employs a different dose and/or time course, etc.

The ability of PAs to trigger the lysis of clots has led to the use of PAs and other plasminogen-activating proteases such as streptokinase as thrombolytic agents for the treatment of myocardial infarction and stroke, as mentioned above. However, studies have suggested that tPA, which is released by neurons following excitotoxicity such as occurs in ischemia, could increase neuronal damage. Furthermore, release or leakage of tPA out of the vascular system and the attendant potential for damage to nervous system tissue, is a recognized hazard of thrombolytic therapy. Thus the invention described herein, which demonstrates that appropriate administration of plasmin and/or plasminogen-activating proteases such as tPA, can actually contribute to structural and/or functional nervous system reorganization and recovery is particularly noteworthy.

It will be appreciated that various embodiments of the present invention differ from previously reported uses of tPA (e.g., for purposes of thrombolysis) in at least one of the following ways, which are described in further detail below: (i) Administration as described herein is focally directed to the nervous system and does not take place via the vascular system; (ii) Administration as described herein is typically performed at least 3 hours following the onset of a stroke or other damaging event and typically at least 12 hours or more following the onset of the damaging event. (iii) Administration as described herein may occur multiple times (e.g., 2, 3, or more times) following the onset of a damaging event and/or may occur either intermittently or continuously over a prolonged time period following the onset of a damaging event (e.g., over at least 1 week, 1 month, 3 months, 6 months, 1 year, 2 years, 3 years, or even longer); (iv) Administration as described herein typically does not use doses that would be sufficient to cause effective blood clot lysis at the site of administration when adminstered using methods that are intended to achieve blood clot lysis.

IV. Methods of Preparing the Agents of the Invention

The agents disclosed herein are all known in the art, and it is believed that appropriate methods for their manufacture are well within the skill of those in the art and therefore need not be described here in detail. For example, and without limitation, tPA, or other polypeptides such as plasmin, growth factors, etc., for use in the present invention, may be purified from natural sources, manufactured using recombinant DNA technology (e.g., recombinant tPA), synthesized using purely chemical synthesis (i.e., synthesis not requiring the use of cells to produce the polypeptide), etc. For example, human tPA is produced and secreted by a number of different melanoma cell lines and can be purified from the cells and/or culture medium (Rijken and Collen, 1981; Bizik, et al., 1990; U.S. Pat. Nos. 4,752,603 and 4,853,330, etc.). Plasmin can be purified from blood or produced recombinantly. If desired, tPA or plasminogen can be cleaved in vitro.

Methods for producing a polypeptide of interest such as tPA, plasmin, growth factors, etc., using recombinant DNA technology are well known in the art. Briefly, such methods generally involve inserting a coding sequence for the polypeptide into an expression vector, operatively associated with expression signals such as a promoter, such that mRNA encoding the protein is transcribed when the expression vector is introduced into a suitable host cell. The host cell translates the mRNA to produce the polypeptide. The polypeptide can include a secretion signal sequence so that the polypeptide is secreted into the medium. The polypeptide may be harvested from the cells or from the medium. Certain suitable methods for preparing tPA using recombinant DNA technology are described in the afore-mentioned U.S. patents, but it is noted that any appropriate method can be used. For example, transgenic animals and plants are commonly used to produce polypeptides. Plants infected by viral vectors are also used to produce polypeptides.

Small molecules such as non-peptide neurotransmitters and analogs thereof, small peptides, neurally active metals, and other compounds disclosed herein are typically either purified from natural sources or chemically synthesized, as appropriate, according to standard methods.

Any of the agents disclosed herein can be provided as pharmaceutically acceptable salts, prodrugs, etc. Furthermore, any of the polypeptides disclosed herein can be modified using a variety of methods known in the art. For example, they can be modified by addition of polyethylene glycol (PEG) or variants thereof (see, e.g., ). Such modifications may increase the active half-life of the polypeptide. See, e.g., Nektar Advanced Pegylation 2005-2006 Product Catalog, Nektar Therapeutics, San Carlos, Calif., which describes a number of such modifying agents and provides details of appropriate conjugation procedures. For administration by injection or infusion, compositions of the invention will typically be mixed with pharmaceutically acceptable carriers or diluent such as sodium chloride (e.g., 0.9%) or dextrose (e.g., 5% dextrose) aqueous solutions. The agents can be provided for administration either in solution or in lyophilized or otherwise dried form. They can be reconstituted in water, saline, etc., followed by dilution in an appropriate pharmaceutically acceptable carrier or diluent.

V. Polymer-Based Drug Delivery Devices

The invention provides a drug delivery device for implantation into the nervous system of a subject to promote functional recovery following ischemic, hemorrhagic, neoplastic, or traumatic damage to the nervous system. The drug delivery device comprises a release material, a proteolysis-enhancing agent and, optionally, one or more additional active agent. The term “release material” is used to refer to any matrix or material that releases incorporated molecules by diffusion or disintegration of the matrix. In certain embodiments of the invention the release material is a biocompatible polymer. The proteolysis-enhancing agent is released from the release material in an amount effective to promote reorganization and/or recovery of the nervous system. A drug delivery device in which an an active agent is physically associated with a polymeric material such as those disclosed herein is referred to as a “polymer-based drug delivery device” in order to distinguish such devices from mechanical drug delivery devices such as infusion pumps, which are used in various embodiments of this invention, though it should be recognized that materials other than polymers could also be used.

In certain embodiments of the invention the proteolysis-enhancing agent is incorporated into or otherwise physically associated with a biocompatible polymeric matrix, which may be biodegradable or nonbiodegradable. Any form of physical association is acceptable provided that the association remains stable under conditions of storage and implantation and for sufficient time to release the active agent over a desired time period. For example, the active agent may be encapsulated within a polymeric matrix, entrapped or entangled within a polymeric matrix, adsorbed to the surface of a polymeric matrix etc. The matrix is delivered to or implanted into the body at the location of the target tissue or in the vicinity thereof. The agent is released from the polymeric matrix over a period of time, e.g. by diffusion out of the matrix or release into the extracellular environment as the matrix degrades or erodes.

The polymeric matrix may assume a number of different shapes. For example, microparticles of various sizes (which may also be referred to as beads, microbeads, microspheres, nanoparticles, nanobeads, nanospheres, etc.) can be used. Polymeric microparticles and their use for drug delivery are well known in the art. Such particles are tyically approximately spherical in shape but may have irregular shapes. Generally, a microparticle will have a diameter of less than 500 microns, more typically less than 100 microns, and a nanoparticle will have a diameter of 1 micron or less. If the shape of the particle is irregular, then the volume will typically correspond to that of microspheres or nanspheres. Methods for making microspheres are described in the literature, for example, in U.S. Pat. No. 4,272,398, Mathiowitz and Langer, 1987; Mathiowitz et al, 1987; Mathiowitz et al., 1988; Mathiowitz et al., 1990; Mathiowitz et al., 1992; and Benita et al., 1984. Solid nanoparticles or microparticles can be made using any method known in the art including, but not limited to, spray drying, phase separation, single and double emulsion solvent evaporation, solvent extraction, and simple and complex coacervation. Preferred methods include spray drying and the double emulsion process. Solid agent-containing polymeric compositions can also be made using granulation, extrusion, and/or spheronization.

The conditions used in preparing the microparticles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the polymer matrix. If the particles prepared by any of the above methods have a size range outside of the desired range, the particles can be sized, for example, using a sieve.

Solid nanoparticles or microparticles can be suspended or dispersed in a pharmaceutically acceptable fluid such as physiological saline and focally administered by injection or infusion (e.g., using a pump) to the nervous system.

Solid polymer-agent compositions (e.g., discs, wafers, tubes, sheets, rods, etc.) can be prepared using any of a variety of methods that are well known in the art. For example, in the case of polymers that have a melting point below the temperature at which the agent is to be delivered and/or at which the polymer degrades or becomes undesirably reactive, a polymer can be melted, mixed with the agent to be delivered, and then solidified by cooling. A solid article can be prepared by solvent casting, in which the polymer is dissolved in a solvent, and the agent is dissolved or dispersed in the polymer solution. Following evaporation of the solvent, the substance is left in the polymeric matrix. This approach generally requires that the polymer is soluble in organic solvent(s) and that the agent is soluble or dispersible in the solvent. In still other methods, a powder of the polymer is mixed with the agent and then compressed to form an implant. Microparticles or nanoparticles comprising a polymeric matrix and a proteolysis-enhancing agent and optionally one or more other active agents can be compressed, optionally with the use of binders, to form an implant.

A polymeric matrix can be formed into various shapes such as wafers, tubes, discs, rods, sheets, etc., which may have a range of different sizes and volumes. For example, prior to polymerization a polymer solution may be poured into a mold having the appropriate shape and dimension. Following polymerization the material assumes the shape of the mold and is usable as an implant. The agent(s) may be present in the solution prior to polymerization, or the implant may be impregnated with the agent following its fabrication.

Suitable biocompatible polymers, a number of which are biodegradable include, for example, poly(lactides), poly(glycolides), poly(lactide-co-glycolides), poly(lactic acids), poly(glycolic acids), poly(lactic acid-co-glycolic acids), polycaprolactone, polycarbonates, polyesteramides, polyanhydrides, poly(amides), poly(amino acids), polyethylene glycol and derivatives thereof, polyorthoesters, polyacetals, polycyanoacrylates, polyetheresters, poly(dioxanone)s, poly(alkylene alkylates), copolymers of polyethylene glycol and polyorthoesters, biodegradable polyurethanes. Other polymers include poly(ethers) such as poly)ethylene oxide), poly(ethylene glycol), and poly(tetramethylene oxide); vinyl polymers-poly(acrylates) and poly(methacrylates) such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; poly(siloxanes), etc. Other polymeric materials include those based on naturally occurring materials such as polysaccharides (e.g., alginate), chitosan, agarose, hyaluronic acid, gelatin, collagen, and/or other proteins, and mixtures and/or modified forms thereof. Chemical derivatives of any of the polymers disclosed herein (e.g., substitutions, additions of chemical groups, for example, alkyl, alkylene, hydroxylations, oxidations, and other modifications routinely made by those skilled in the art) are also encompassed. Furthermore, blends, graft polymers, and copolymers, including block copolymers of any of these polymers can be used. It will be appreciated that a vast number of different polymer variations are available. It will be understood that certain of these polymers require use of appropriate initiators or cross-linking agents in order to polymerize.

One of skill in the art will understand that in choosing an appropriate polymer and method of manufacture, it is important to select materials and methods that are compatible with stability of the agent. For example, it may be desirable to avoid processing temperatures that are likely to result in substantial degradation or denaturation of the agent, which may result in loss of bioactivity. It will also be desirable to test the composition so as to ensure that the agent is released in significant amounts over the desired period of time.

In general, the following criteria are important for selection of a material to be used for delivery of the active agent(s): (1) minimal or no cytotoxicity, (2) minimal or no elicitation of immune responses and inflammation, (3) compatibility with aqueous solutions and physiological conditions, (4) compatibility of the material and its processing methods with the stability of theagent to be incorporated. It may be desirbable to utilize a material with a controlled rate of biodegradation. Features such as cross-linking and monomer concentration may be selected to provide a desired rate of degradation and release of the agent. It will be appreciated that a polymeric drug delivery device of the invention may include one or more pharmaceutically acceptable materials such as buffers, spheronizing agents, fillers, surfactants, disintegrants, binders, or coatings. Exemplary materials are described in U.S. Pat. No. 5,846,565.

Methods for purifying or synthesizing the various polymers for use in drug delivery systems of the invention are known in the art. Methods for incorporating therapeutically active agents into polymeric matrices are likewise known in the art. For example, the active agent can be combined in solution with the polymer prior to polymerization or can be provided in solid form and encapsulated as the polymer polymerizes. A number of different agents have been delivered to the CNS using such polymer matrices. For example, chemotherapeutic agents have been delivered to tumors in the nervous system by encapsulating the agent in a polymeric matrix, which is made into a shaped form, and surgically implanting the matrix into the brain. See, e.g., U.S. Pat. Nos. 5,626,862; 5,651,986; and 5,846,565. Additional drug delivery devices in which an active agents is provided in a polymeric matrix are described, e.g., in U.S. Pat. Nos. 4,346,709; and 5,330,768. See also, Wu, 1994; Dang, 1996; Fleming, 2002; Westphal, 2002.

Similar methods to those used in the afore-mentioned references are of use to focally deliver the agents of the invention. In certain embodiments of the invention the drug delivery device provides controlled or sustained release, i.e., the proteolysis-enhancing agent and any other agents contained in the device are released over a prolonged period of time, e.g., hours to days, weeks, or months.

Preparation of polymer-agent drug delivery devices can be performed using standard methods known in the art. Briefly, drug delivery devices are typically prepared in one of several ways. For example, the polymer can be melted, mixed with the substance to be delivered, and then solidified by cooling. Such melt fabrication processes generaly utilize polymers having a melting point that is below the temperature at which the substance to be delivered and the polymer itself degrade or become reactive. Alternatively, the device can be prepared by solvent casting, where the polymer is dissolved in a solvent, and the substance to be delivered dissolved or dispersed in the polymer solution. The solvent is then evaporated, leaving the substance in the polymeric matrix. Solvent casting typically utilizes a polymer that is soluble in organic solvents, and the drug to be encapsulated should be soluble or dispersible in the solvent. Similar devices can be made by phase separation or emulsification or even spray drying techniques. In still other methods, a powder of the polymer is mixed with the agent and then compressed to form an implant.

Methods of producing implants also include granulation, extrusion, and spheronization. A dry powder blend is produced including the desired excipients and microspheres. The dry powder is granulated with water or other non-solvents for microspheres such as oils and passed through an extruder forming “strings” or “fibers” of wet massed material as it passes through the extruder screen. The extrudate strings are placed in a spheronizer which forms spherical particles by breakage of the strings and repeated contact between the particles, the spheronizer walls and the rotating spheroniter base plate. The implants are dried and screened to remove aggregates and fines.

These methods can be used to make microimplants (microparticles, microspheres, and microcapsules encapsulating drug to be released), slabs or sheets, films, tubes, and other structures. A preferred form for infusion or injection is microimplants, as described elsewhere herein.

Proteins and peptides have been successfully incorporated into polymeric matrices. For example, insulin has been incorporated into biodegradable polymeric microcapsules and retains essentially the same bioactivity as the free form (Takenaga 2004). Natural and synthetic collagenous matrices have been used as carriers of a variety of different growth factors (Kanematsu, 2004).

Of particular interest in the present invention are polymers that form hydrogels, i.e., gels that contain a substantial proportion of water. Hydrogels may, for example contain 50%, 60%, 70%, 80%, 90%, or an even greater amount of water on a w/w basis. Polymeric materials can be formed into hydrogels either prior to or following administration to a subject. As described in Example 5, the inventors produced hydrogel discs comprising hPLA-b-PEG-PLA macromers. The discs were formulated to incorporate tPA by mixing tPA with the polymer solution prior to initiating polymerization. Experiments showed that the hydrogel discs released active tPA over a period of 10 days (the length of the experiment). Other suitable hydrogel-forming polymers are known in the art.

The polymer-based drug delivery devices of the invention may be implanted at any desired location within the CNS. For example, and without limitation, the polymer-based drug delivery device can be implanted either in the brain (e.g., close to a site of damage such as an ischemic region following stroke, or in the opposite brain hemisphere), or in the base of the brain, in or near a CSF-filled space such as ventricle, etc. In the case of a device implanted into a CSF-filled space, the device releases the agent into the CSF, allowing it to diffuse to a region of the brain surround the space. Depending on the size of the device, it can also be implanted at or adjacent to a nerve, nerve tract, ganglion, etc., of the PNS. For example, microimplants can be implanted within or internal to the epineurium or perineurium of a nerve.

VI. Implantable Microchip-Based Delivery

In certain embodiments of the invention, one or more agent(s) is delivered to the nervous system using an external or implantable silicon or polymeric microchip, which contains from dozens to up to hundreds or thousands of microreservoirs, each of which can be filled with any combination of drugs, reagents, or other chemicals. The micro-reservoirs can be opened at predetermined times and/or on demand using preprogrammed microprocessors, remote control, or biosensors. If desired, complex chemical release patterns can be achieved using these approaches. In other embodiments the micro-reservoirs have a “cap” that degrades over time. Release can be controlled by varying the thickness and/or composition of the cap, thereby allowing release to occur at predictable and substantially predetermined times. The cap material can be, e.g., a degradable polymer. In other embodiments the cap material is non-degradable and is permeable to the molecules to be delivered. The physical properties of the material used, its degree of crosslinking, and its thickness will determine the time necessary for the molecules to diffuse through the cap material. If diffusion out of the release system is limiting, the cap material delays release. If diffusion through the cap material is limiting, the cap material determines the release rate of the molecules in addition to delaying release time.

In some embodiments the agent(s) to be delivered are inserted into the reservoirs in their pure form, as a liquid solution or gel, or they may be encapsulated within or by a release material. The release material may be, for example, a biodegradable or non-biodegradable polymer. Representative polymers include those mentioned above. See, e.g., Santini, J T, et al., 2000; U.S. Pat. Nos. 5,797,898; 6,808,522, and U.S. Pub. Nos. 20020072784 20040166140; 20050149000, for discussion of microchip-based delivery systems. Microchips can be implanted at any desired location in the CNS (as described above). Depending on the size of the device, it can also be implanted at or adjacent to a nerve, nerve tract, ganglion, etc., of the PNS. For example, microchips can be implanted within or internal to the epineurium or perineurium of a nerve.

VII. Methods for Focal Delivery

In accordance with the invention, a composition comprising a proteolysis enhancing agent such as tPA, plasmin, plasminogen, or another agent described herein such as an agent that enhances tPA or plasmin synthesis, an agent that inhibits a PAI, etc., is administered to a subject by focal delivery. Focal delivery may be accomplished in a number of different ways. Implantation of a polymer-based drug delivery device or microchip such as those described above at a site within the central nervous system or within or adjacent to a nerve, nerve tract, or ganglion within the peripheral nervous system is a suitable method to achieve focal delivery.

Internal (implantable) or external pumps can be employed for administering a substantially fluid composition of the invention. Such pumps typically include a drug reservoir from which continuous or intermittent release occurs into the target tissue or in the vicinity thereof via a catheter. In certain embodiments of the invention treatment is carried out using an implantable pump and a catheter having a proximal end coupled to the pump and having a discharge portion for infusing therapeutic dosages of one or more agents described herein into a predetermined infusion site in brain tissue or into the spinal canal (intrathecal delivery).

Infusion (which term is used to refer to administration of a substantially fluid material to a location in the body by means other than injection) may be carried out in a continuous or nearly continuous manner, or may be intermittent. The pump may be programmed to release predetermined amounts of the agent at predetemined time intervals. U.S. Pat. No. 4,692,147, assigned to Medtronic, Inc., Minneapolis, Minn., describes a suitable pump. In certain embodiments one or more of the infusion systems known as the Synchromed® Infusion System manufactured by Medtronic, Inc. of Minneapolis, Minn. (see web site having URL www.medtronic.com) is used. However, it will be appreciated that the pump may take the form of any device used for moving fluid from a reservoir. Mechanical, pressure-based, osmotic, or electrokinetic means may be used.

In order to deliver an agent to the brain parenchyma, a catheter attached to the pump may be implanted so that the discharge portion lies in the brain parenchyma. See, e.g., U.S. Pat. No. 6,263,237 for description of a variety of suitable systems and methods for implanting them into the body of a subject and directing the administration of an active agent to a desired location in the brain. Continuous ICM, is a relatively new technique of regional delivery of therapeutic agents directly into brain parenchyma, which establishes a bulk flow current that has the potential to homogeneously distribute even large molecules. See e.g., Laske, 1997, for an example of administration of an agent to a region within the brain.

In certain embodiments of the invention the agent is delivered to one or more of the CSF-containing cavities or chambers of the central nervous system, e.g., the ventricles or cisterna magna, which is located at the bottom of the skull. As is well known in the art, there are two lateral ventricles and midline third and fourth ventricles within the brain. To deliver an agent to a ventricle or the cistema magna using an infusion pump, the catheter may be implanted so that the discharge portion lies in the ventricle or the cisterna. The agent diffuses out of the ventricle or cistema magna. Delivery to these locations therefore allows delivery of the agent to a relatively wide area of the brain rather than localizing it more closely to a specific site. Intraventricular or intracisternal administration is considered to be administration to the nervous system. In certain embodiments of the invention delivery to a CSF-containing space, e.g., a ventricle, is accomplished by surgically implanting a catheter through the skull so that the tip has access to the space. The other end of the catheter is then connected to a reservoir (e.g,. an Ommaya reservoir), which is placed beneath the scalp (subcutaneously). This method is in use for delivery of chemotherapeutic agents. See, e.g., Ommaya and Punjab, 1963; Galicich and Guido, 1974; Machado, 1985; Obbens, 1985; Al-Anazi, 2000.

If the subject suffers from damage to the spinal cord, the catheter is implanted so that the discharge portion lies in an intrathecal space of the spinal cord while the other end is connected to the pump reservoir. Methods for administering agents to the spinal fluid (i.e., intrathecally) are well known in the art. Such methods are commonly used in the treatment of chronic pain, and are routinely used to deliver analgesic agents over a period of months. Similar methods are of use in the present invention. See, e.g., Lamer, 1994; Paice, 1996; Winkemuller, 1996; Tutak, 1996; and Roberts, 2001 for descriptions of the use of implantable pumps for delivery of a variety of different therapeutic agents for treatment of a number of different conditions.

For delivery to the PNS, suitable methods include injection or infiltration into a nerve or nerve trunk, e.g., adjacent to a site of nerve damage, and implantation of a polymer-based delivery device or microchip either adjacent to a site of nerve damage. Methods for administering anesthetic agents to diverse nerves, nerve bundles, etc., within the PNS are well known in the art, and any of these methods are applicable in the context of the present invention.

In certain embodiments of the invention a solution comprising a polymer, a proteolysis-enhancing agent, and optionally one or more additional active agents is administered by injection or infusion using any of the means described above. The polymer assembles to form a gel upon administration, e.g., following contact with physiological fluids. Such assembly may, for example, be triggered by exposure to monovalent or divalent cations. For example, U.S. Pub. No. 20020160471 describes self-assembling peptides that form hydrogels. U.S. Pat. No. 6,129,761 describes a variety of different self-assembling polymers and polymers that require a polymerizing agent or cross-linking agent to faciliatate assembly. Certain of these polymers assemble to form hydrogel stuctures upon contact with physiological fluids following administration to a subject. In another embodiment a collagen-based system is used. See, e.g., WO 00/47130, which describes injectable collagen-based systems for delivery of cells or therapeutic agents.

VIII. Delivery Location, Timing. Duration of Treatment, and Dose

The location at which a composition of the invention is to be administered or implanted is selected with relation to the particular condition being treated. For example, if the subject has suffered an injury or damage to the brain, e.g., as a result of stroke, trauma, etc., the composition may be delivered to the brain parenchyma or to one or more of the ventricles of the brain or to the cistema magna. If the subject has suffered an injury or damage to the spinal cord, a composition of the invention is delivered to the spinal cord, e.g, by implanting or administering a composition within the spinal canal.

The area to which the agent is to be administered may be, for example, an area that has been damaged (e.g., an ischemic lesion) or an area adjacent to an area that has been damaged. The agent(s) may be administered to any region, nucleus, or functional area within the brain including, but not limited to, any of the major subdivisions of the brain (cortex, hippocampus, cerebellum, thalamus, midbrain, brain stem), which include motor cortex, sensory cortex including visual cortex, auditory cortex, and somatosensory cortex, language areas of cortex, frontal cortex, internal capsule, basal ganglia, thalamus, etc. Numerous specific areas within the brain have been defined based on anatomical and histological considerations. In addition, areas in the brain that are responsible for performing various tasks have been defined on functional grounds and are well known in the art. See, e.g., Kandel, supra and Victor and Ropper, supra.

In certain embodiments of the invention the area that has been damaged is identified. The area that has been damaged can be identified using a variety of different imaging techniques known in the art. For example, and without limitation, suitable methods include imaging techniques such as magnetic resonance imaging (MRI), optionally imaging features associated with blood flow such as perfusion, diffusion, or both, computed tomography (CT), positron emission tomography (PET), ultrasound, etc. Imaging techniques that image structure and/or function are available. Functional studies can be performed, e.g., using labeled substrates such as glucose to identify regions of the brain that are metabolically inactive and/or that do not respond to stimulation, suggesting that they are functionally inactive. See, e.g., Grossman and Yousem, supra.

Clinical diagnosis can be used instead of, or in addition to, imaging techniques. For example, the area to which damage has occurred can be identified by performing a neurological examination. Deficits noted on the neurological examination can be correlated with damage to particular areas of the central and/or peripheral nervous system (Kandel, supra; Victor and Ropper, supra).

Any of the foregoing methods can be utilized acutely (e.g., within hours to a few days of a damaging event such as stroke or injury) or at later times, e.g., several days to weeks, months, or years following the event. The characteristic evolution of the appearance of nervous system lesions is well known in the art, so the practitioner can readily identify the location of damaged tissue at any desired time point relative to the time at which the event causing the damage occurred.

In certain embodiments of the invention the agent is delivered at or adjacent to a site where tissue necrosis and/or scar tissue formation has occurred in the CNS. Areas of necrosis can be identified using various imaging techniques such as those mentioned above. Symptoms may also be used to guide selection of an appropriate location at which to implant the matrix. For example, if a subject experiences impairment of a particular function such as movement, sensation, speech, etc., then the portion of the brain that is normally responsible for control or achievement of that function, or the corresponding area on the contralateral side of the subject's body, may be selected as a suitable site for implantation of a drug delivery device of the invention. Standard surgical techniques can be used.

In some embodiments of the invention the agent is administered to an area adjacent to a region that has been damaged by an infarct, e.g., to the peri-infarct area. Without wishing to be bound by any theory, peri-infarct regions are likely to be sites of clinically relevant cortical remodeling following stroke. For example, the agent may be administered to a site that is located up to approximately 0.5 cm from the edge of an infarcted area, up to 1.0 cm from the edge of an infarcted area, or up to 2 cm from the edge of an infarcted area. In some embodiments the agent is administered to a site immediately adjacent to an infarcted area, e.g, up to 0.5 cm from the edge of the infarcted area. In some embodiments of the invention the agent is administered to the ischemic penumbra adjacent to an area of severe ischemia following stroke. See, e.g., Furlan, M., et al., 1996. The ischemic penumbra is a region of brain tissue that experiences mild to moderate ischemia but remains viable for a period of time following a stroke (e.g., up to several hours or longer) and may be salvageable if perfusion is re-established and/or through the use of neuroprotective agents. The ischemic penumbra may be operationally defined using, e.g., diffusion and perfusion MRI (Schlaug, G., et al., 1999; Kidwell, C. S., et al. 2003). One of ordinary skill in the art will be able to select an appropriate definition and measurement technique.

In some embodiments of the invention the agent is administered to a location on the opposite side of the brain from the side where damage has occurred. The site of administration may be substantially symetrically located with respect to the region that has been damaged. Without wishing to be bound by any theory, it is possible that following damage to a particular region of the brain, the contralaterally located region reorganizes so as to assume responsibility for functions that were previously performed by the damaged region. For example, a portion of the brain that normally (prior to injury) generates movement commands for the left hand only may reorganize so as to generate commands to both hands following damage to a portion of the brain that previously commanded the right hand.

As mentioned above, delivery by injection or infusion pump is suitable for compositions in which an agent of the invention is dissolved in a liquid and for compositions comprising microparticles of suitable dimensions. The polymer-based drug delivery devices of the invention will typically be implanted into the subject in an appropriate location in the nervous system so that they will release the active agent at a desired location. For example, they may be implanted into the brain parenchyma. They may also be implanted into a ventricle or into the spinal canal in various embodiments of the invention. The location for implantation is selected so as to achieve an effective concentration of the active agent at a desired location in the nervous system, i.e., typically reasonably close to the location at which it is desired to achieve the effective concentration. Care is taken to avoid disrupting undamaged portions of the nervous system to the extent possible. Imaging may be used to guide administration or implantation of the compositions and drug delivery devices of the invention, e.g., they may be administered or implanted under stereotactic guidance.

The agent can be administered in a continuous or intermittent fashion. Intermittent or pulsatile delivery may be performed at times selected in accordance with the active half-life of the agent in order to maintain a therapeutically useful dose and/or may be performed in accordance with physiological patterns such as circadian rhythms, or during periods when the subject either is or is not engaged in particular activities. If the agent is administered using an implanted device such as a pump or microchip, an external controller may be used to trigger release at a desired time, or the device can be programmed to release the agent at particular times or intervals.

The compositions of the invention will typically be administered at least 3 hours after the onset or occurrence of a damaging event such as a stroke or injury. For example, the initial administration may be at least 12, 24, 36, or 48 hours after the onset or occurrence of a damaging event. In certain embodiments of the invention the initial administration is between 24 hours and 1 week after the onset or occurrence of a damaging event, between 1 week and 1 month after the onset or occurrence of a damaging event, or between 1 and 3 months, 3 and 6 months, 6 and 12 months after the onset or occurrence of a damaging event, etc. The initial administration may occur at times greater than 1 year following the onset or occurrence of a specific damaging event, e.g., 2-5 years, etc. In some embodiments of the invention the initial administration occurs after the subject has reached a plateau of functional recovery. For example, the subject may have failed to display improvement on one or more standardized tests, or may have failed to experience subjective improvement during the preceding 1-3 months, 3-6 months, or longer. For treatment of neurodegenerative diseases, nutrient deprivation, neoplastic diseases, and other conditions for which there is no specific damaging event, administration can occur at any time following diagnosis of the disease.

The total time period during which treatment occurs, and the number of treatments within such time period, can vary. The total duration of treatment (i.e., the time interval between the first and the last treatment) can range from days to weeks, months, or years. For example, the total duration may be 1, 3, 6, 9, or 12 months, between 1 and 2 years, 2 and 5 years, etc. If the agent is administered in discrete doses in addition to or instead of being administered continuously, subjects may receive anywhere from a single dose to dozens or even hundreds or thousands of doses. The time interval between doses can be varied. It may, for example, be desirable to provide the agent for a defined time period each day, e.g., 10 minutes/day, 1 hr/day, etc.

The effective dose of the proteolysis-enhancing agent to be administered will be selected taking into account the particular agent, the condition being treated, and other relevant factors. The dose (or doses) may be, e.g., an amount effective to promote growth or sprouting of axons, promote structural reorganization of synaptic connections, increase formation of new synaptic connections, increase dendritic spine motility, inhibit structural or functional degeneration (e.g., degeneration that would otherwise be expected to take place) or any combination of the foregoing. Typically the dose for each administration of the agent will be significantly lower than the dose that would be required to cause lysis of a significant blood clot when administered to the vascular system. Exemplary, non-limiting doses ranges for an agent of the invention, e.g., tPA, include one or more of the following: (i) a dose sufficient to achieve a concentration of between 10 and 100,000 IU/ml or between 100 and 10,000 IU/ml or between 100 and 1,000 IU/ml in the extracellular fluid or in a CSF-containing cavity such as a ventricle or the spinal canal; a dose between 1 μg/day and 10 mg/day; a dose between 1 μg/day and 1 mg/day; a dose 5 μg/day and 500 μg/day; a dose between 10 μg/day and 100 μg/day, etc.

Various dosing regimens may be used. For example, it may be desirable to give a relatively large “loading dose” initially and then administer smaller doses either continuously or intermittently so as to maintain an effective concentration in the region of the nervous system being treated. It will also be appreciated that, in general, the more focally directed the delivery, the lesser the total dose that may be required. Thus direct administration via a catheter to a specific brain region may require a lower total dose than delivery to a ventricle. Furthemore, the larger the area of damage and/or the greater the amount of reorganization and/or recovery required, the larger might be the dose.

As described in Example 3, a dose of 362.5 IU/ml for 45 minutes was effective in promoting structural plasticity in neurons, while doses of 2 μg/day (1450 IU/day) and 10 μg (7250 IU/day) were effective in animal models (rats) and suggest that doses in the 1-1000 microgram/day range may be suitable in certain embodiments of the invention. These low doses contrast with the dose of 100 mg/3 hr (58,000,000 IU/3hr) of Alteplase administered to human subjects for purposes of thrombolysis.

If desired, the concentration of the proteolysis-enhancing agent (or any other agent whose administration is contemplated in the present invention) can be monitored, e.g,. in the CSF of the subject. The dose can be adjusted accordingly to obtain a desired concentration.

In certain embodiments of the invention the agent is administered, e.g., released, in a defined temporal relation to rehabilitative therapy, e.g., during, prior to, or following engagement of the subject in one or more rehabilitative activities. The agent may, for example, be administered up to 5 minutes −12 hours prior to the activity, up to 5 minutes −12 hours after the activity, during the activity, or immediately prior to or immediately following the start of a therapy session, e.g,. up to 5 minutes prior to the beginning of a therapy session or up to 5 minutes following the start of a therapy session. By “therapy session” is meant any period of time in which the subject is engaged in performing activities that have been suggested or prescribed by a health care provider for purposes of assisting the functional recovery of the subject following damage to the CNS or PNS. The health care provider need not be present during the therapy session, e.g., the subject may perform the activities independently or with the assistance of personnel other than a health care provider.

IX. Administration of Additional Active Agent(s). Cells, and Gene Therapy

In various embodiments of the invention one or more additional active agents is administered to the subject in conjunction with administration of the proteolysis-enhancing agent. By “in conjunction with” is meant either concurrently or sequentially. The additional active agent may be delivered focally but may alternatively be administered systemically using any suitable route of administration (e.g., oral, intravenous, intramuscular, subcutaneous, transdermal, pulmonary, etc.). The substance may be delivered in the same solution or dosage form as the proteolysis-enhancing agent. The substance may be incorporated into a polymeric matrix together with the proteolysis-enhancing agent and delivered via a polymer-based drug delivery device or delivered using a pump or any other delivery system disclosed herein.

In some embodiments of the invention an agent other than a proteolytic agent is administered instead of, or in conjunction with, a proteolysis-enhancing agent such as those described above, wherein the agent cleaves one or more components of the extracellular matrix at a bond other than a peptide bond. For example, the agent may cleave a polysaccharide portion of an ECM component such as a proteoglycan or glycosaminoglycan. Examples of suitable agents include chondroitinases (which cleave chondroitin sulfate and hyaluronic acid), hyaluronidases, heparinases (which cleave heparin), heparanase (which cleaves heparan sulfate), etc.

In certain embodiments of the invention the additional active agent is a neural growth enhancing agent. A neural growth enhancing agent is any molecule or cell that promotes, enhances, increases, etc., one or more aspects of the growth or regeneration of neural tissue. For example, the molecule or cell may promote axon growth. A neural growth enhancing agent, as used herein, can be a neurally active growth factor, neurotransmitter or neurotransmitter analog, neurally active metal, modulator of a synaptic signaling molecule, or cell. It will be understood that typically “cell”, as used in this context, refers to multiple cells. The term “neurally active” means that the agent exerts a biological effect on neural tissue. For example, the agent may exert an effect that enhances structural and/or functional nervous system reorganization or recovery.

The invention therefore provides a composition comprising a proteolysis-enhancing agent and a neural growth enhancing agent. The proteolysis-enhancing agent can be, for example, one or more of any of the proteolysis-enhancing agents disclosed herein or known in the art. The neural growth enhancing agent can be, for example, one or more of any of the neural growth enhancing agents disclosed herein or known in the art. The invention provides a drug delivery device comprising the composition. The drug delivery device can be, for example, any of the drug delivery devices described herein.

The invention further provides a method for promoting recovery or reorganization in the nervous system of a subject comprising the step of: administering a a proteolysis-enhancing agent and a neural growth enhancing agent to a subject in need of enhancement of recovery or reorganization of the nervous system. The subject is typically in need of recovery or reorganization of the nervous system as a result of ischemic, hemorrhagic, neoplastic, degenerative, or traumatic damage to the nervous system. The invention further provides a method of treating a subject in need of enhancement of recovery or reorganization in the nervous system comprising the step of: administering a proteolysis-enhancing agent and a neural growth enhancing agent to the subject. The subject is typically in need of enhancement of recovery or reorganization of the nervous system as a result of ischemic, hemorrhagic, neoplastic, degenerative, or traumatic damage to the nervous system. Either or both of the agents in the afore-mentioned methods can be administered focally to the central or peripheral nervous system either individually or in combination using any of the methods described herein. Either or both of the agents can be administered by any alternate route of administration. Certain features of this aspect of the invention, e.g., dose ranges for the proteolysis-enhancing agent, adjunct therapy, etc., can be similar to those described for other aspects of the invention.

Neurally active growth factors include, but are not limited to, nerve growth factor (NGF), brain-derived neurotrophic factor (BDNF), neurotrophin-1 (NT-3), neurotrophin-4/5 (NT-4/5), ciliary neurotrophic factor (CNTF), leukemia inhibitory factor (LIF), glial cell derived growth factor (GDNF), neurturin, artemin, persephin, insulin-like growth factor 1 (IGF-1), acidic or basic fibroblast growth factor (aFGF, bFGF), osteogenic protein-1 (OP-1), vascular endothelial growth factor (VEGF), erythropoietin (EPO), and granulocyte colony stimulating factor (G-CSF).

“Synaptic signaling molecules” refers to endogenous molecules that are activated downstream of calcium entry into cells through synaptic activation or following release of calcium from intracellular stores and that transduce electrical activity into structural changes in neurons. These include a variety of kinases such as calcium/calmodulin-dependent protein kinase II and IV, protein kinase C, protein kinase A, extracellular signal regulated kinase (ERK), cyclic AMP dependent kinase, along with molecules such as cyclic AMP response element binding protein (CREB), activity regulated cytoskeletal associated protein (arc), troponin C, and Rac and Rho pathways and their associated kinases. G protein coupled receptors transduce information from the extracellular space to intracellular signals (among other activities) and are also considered to be synaptic signaling molecules. Modulators (i.e., agents that activate or inhibit) of a number of these signaling molecules are known in the art and are of use in the present invention. Molecules that can bind to G protein coupled receptors importantly include those that can activate or inhibit (a) protein kinase A (PKA) and cAMP; (b) cyclic GMP, and (c) protein kinase C (PKC). Pathways downstream of GPCR activation importantly regulate CREB, BDNF, actin, reorganization of the dendritic and axonal cytoskeleton, etc. By way of example, activators of cAMP include Sp-cAMPS (Sigma), which may to be delivered into the brain at a typical dose of 0.02-0.5 ug/kg/day, and Rolipram® (Sigma), which can be given intramuscularly at a dose of 1-100 ug/kg/day (Ramos et al., Neuron 2003). Rolipram is a phosphodiesterase inhibitor, which prevents breakdown of cAMP. Inhibition of cAMP can also, under certain conditions, have a stimulatory effect on synapses and is of use in certain embodiments of the invention. Inhibitors of cAMP include Rp-cAMPS (Sigma), which can be delivered into the brain at a typical dose of 0.02-0.5 ug/kg/day (Ramos et al., 2003).

An activator of cGMP is 8-Br-cGMP; an inhibitor is Rp-cGMPs. Both are preferably delivered focally. Effective doses on neurite growth and dynamics in brain slices are about 10-100 uM (Nishiyama et al., 2003). Another inhibitor is ODQ; an effective dose for influencing axon growth is about 10 uM (Leamey et al., 2001). Activators of PKC include diacylglycerol and phosphatidylserine. An inhibitor is a drug called GF109203X (GFX). Effective doses in slices are approximately 10-100 uM (Nishiyama et al., 2003).

It is noted that doses presented here should in no way be considered limiting. In general, the invention encompasses doses at least 10 to 100 fold lower than those described here, and doses up to the maximum tolerated dose of the agent, as consistent with sound medical judgment. Furthermore, dosage routes for specific agents are mentioned here by way of example and are not intended to be limiting. In general, any suitable route of administration can be used. In particular, any of these agents may be administered using the methods for focal administration described herein.

Neurally active small molecules include a number of the modulators and neurotransmitters described above as well as diverse compounds known in the art to influence nervous system function. See, e.g., Goodman and Gilman, supra and Kandel, supra.

One of ordinary skill in the art will readily understand which particular neurally active growth factors and synaptic signaling molecules are referred to using the names listed herein and will be able to retrieve the sequences of these polypeptides and relevant information such as sources from which the molecule can be purified or obtained using, e.g,. publicly available databases such as Genbank and Pubmed. For example, one of skill in the art can search the Entrez Gene database provided by the National Center for Biotechnology Information (NCBI), available at the web site having URL www.ncbi.nlm.nih.gov/entrez/query.fcgi?CMD=search&DB=gene and can thereby locate the Gene ID for any particular gene or protein of interest.

It will be appreciated that the afore-mentioned names may in some cases refer to a family of related molecules, any of which could be used in various embodiments of the invention. For example, the ERK family of kinases contains at least ERK1, ERK2, and ERK3. It will further be appreciated that allelic variants, homologs of the molecule found in particular species, and molecules having substantial sequence identity can also be used. The neurally active growth factor or signaling molecule may, but need not, enhance growth and/or proliferation of neural tissue.

Neurotransmitters are naturally occurring compounds that generally fall into the categories of small molecules (e.g., catecholamines) and peptides. A neurotransmitter for use in the present invention can be excitatory or inhibitory. Exemplary neurotransmitters include, but are not limited to, acetylcholine, dopamine, serotonin, glycine, glutamate, epinephrine, norepinephrine, and gamma aminobutyric acid (GABA). A neurotransmitter analog as used herein is a compound other than a naturally occurring neurotransmitter that exerts an excitatory or inhibitory effect on a neurotransmiter receptor. The analog will typically bear a structural resemblance to a naturally occurring neurotransmitter and will compete with it for binding to its receptor.

Neurally active metals include magnesium and zinc. The magnesium and/or zinc can be provided in any suitable form. Typically the metal will be provided in the form of a salt that contains a metal cation and an anion that serves as a counterion. The counterion can be an organic or inorganic substance. For example, the counterion can be phosphate, carbonate, gluconate, citrate, sulfate, acetate, maltonate, oxalate, or any other pharmaceutically acceptable ion such as those mentioned below. In some embodiments the metal cation is provided as a chelate, in which the metal cation is complexed with an organic molecule such as a heterocyclic ring.

Gene therapy methods may be used to increase expression of genes that encode products, e.g., proteolysis-enhancing agents, that promote nervous system functional and/or structural reorganization and/or recovery. Gene therapy encompasses delivery of nucleic acids comprising templates for synthesis of a molecule of interest to a cell of interest. The nucleic acid (or a nucleic acid derived from the nucleic acid as, for example, by reverse transcription) may be incorporated into the genome of the cell or remain permanently in the cell as an episome. Gene therapy also encompasses delivery of nucleic acids that do not integrate or remain permanently in the cell to which they are delivered. Such approaches permit temporary or transient synthesis of a molecule of interest. Methods and materials for performing gene therapy are well known in the art and will not be extensively reviewed here. See, e.g., Berry, 2001; Han, 2000; and Thomas and Klibanov, 2003.

Vectors and delivery vehicles (e.g., polymeric matrices) that provide nucleic acids comprising templates for synthesis of polypeptides such as tPA, plasmin, or any of the other polypeptides disclosed herein may be incorporated into a composition of the invention or administered separately. Preferably the nucleic acid includes a coding sequence for a gene to be expressed in a cell of interest and also includes appropriate expression signals, e.g., promoters, terminators, etc., to ensure proper expression.

In general, either viral or non-viral vectors may be used. For example, herpes virus, adenovirus, adeno-associated virus, retroviruses, or lentiviruses may be used. It may be preferable to avoid the use of intact viruses in delivering templates to cells. Thus it may be preferable to deliver DNA vectors or linear DNA molecules. These vectors may, but need not, include viral sequences such as long terminal repeats, etc. Any of a wide variety of agents useful for transfection may be used to enhance uptake of nucleic acids by cells. The vectors are taken up by cells in the nervous system, and the polypeptide of interest, e.g., tPA, plasmin, etc., is expressed and, preferably secreted.

In some embodiments of the invention cells are administered to a subject who also receives a composition comprising a proteolysis-enhancing agent. In some embodiments of the invention the cells serve as a source for a proteolysis-enhancing agent. For example, the cells secrete tPA into the extracellular space. In certain embodiments of the invention the cells are genetically modified prior to their administration to increase their synthesis of a proteolysis-enhancing agent such as tPA, plasmin, etc. For example, the cells may be stably transformed with a vector that comprises a template for transcription of an RNA that encodes the proteolysis-enhancing agent. The cells may be sequestered in a preferably non-biodegradable reservoir or compartment that retains them at a particular location and prevents their integration with cells at the site of administration or wider dispersal.

In some embodiments the cells contribute to structural and/or functional recovery of the nervous system. The cells can be neurons, glia, or non-neural cells. Suitable cells include, but are not limited to, Schwann cells and olfactory ensheathing glia (Bunge, 2003). The cells can be of a single cell type, or combinations of different cell types can be administered. The cells may replace or supplement neural tissue that has been irreversibly damaged and/or provide supportive functions. In some embodiments neural stem cells are administered. Multipotent neural stem cells, capable of giving rise to both neurons and glia, line the cerebral ventricles of all adult animals, including humans. In addition, distinct populations of nominally glial progenitor cells, which also have the capacity to generate several cell types, are dispersed throughout the subcortical white matter and cortex (Goldman 2005). In other embodiments adult or embryonic stem cells are administered. Such cells can be derived from a location outside the nervous system, e.g., the bone marrow, liver, umbilical cord, etc. Cells of any type can be used. The cells can be autologous or non-autologous. In certain embodiments the cells are from the same species as the subject.

In certain embodiments of the invention the cells are administered in a polymeric scaffold, made of certain of the materials such as those described above that provide a hospitable environment to maintain cell viability. The polymer material may be biodegradable. The matrix or scaffold may be formed prior to implantation into the nervous system of a subject or may form following administration, e.g., upon contact with physiological fluids. Encapsulation of cells in a variety of different polymeric matrices or scaffolds is well known in the art. See, e.g., U.S. Pat. Nos. 6,129,761; 6,858,229, U.S. Pub. No. 20020160471, and Teng, 2002.

In addition to or instead of the various active agents described above, which are selected primarily based on their useful properties for enhancing structural or functional recovery or reorganization in the nervous system, various other substances can be administered. Such substances include, but are not limited to, antibiotics or antifungal agents to treat or reduce the risk of infection, chemotherapeutic agents to treat tumors, etc.

It is to be understood that the invention explicitly includes compositions comprising each specific combination of any of the proteolysis-enhancing agents described herein and any of the additional active agents described herein. Because it would not be practical to list each and every combination, only a few examples are provided here. For example, the invention includes a composition comprising tPA and a neurally active growth factor (e.g., tPA and BDNF); a composition comprising tPA and a modulator of a synaptic signaling molecule (e.g., tPA and Rolipram);.a composition comprising tPA and a neurotransmitter (e.g., tPA and serotonin); a composition comprising tPA and a neurally active metal (e.g., tPA and magnesium); a composition comprising tPA and a neurally active small molecule; a composition comprising tPA and a cell (e.g., tPA and a neural stem cell), etc. Similarly, the invention includes a composition comprising (i) plasmin and (ii) a neurally active growth factor, a synaptic signaling molecule, a neurotransmitter, a neurally active metal, and/or a cell. Compositions comprising 3, 4, 5, or more of the proteolysis-enhancing agents and/or additional agents are encompassed. The invention further provides a polymer-based drug delivery device comprising any of these compositions and an implantable microchip comprising any of these compositions or designed to administer the agents individually.

The invention further encompasses administration of one or more of any of the proteolysis-enhancing agents described herein in conduction with one or more of any of the additional agents described herein to a subject in need of reorganization and/or recovery of the nervous system. The subject has typically experienced ischemic, hemorrhagic, neoplastic, traumatic, and/or degenerative damage to the central or peripheral nervous system. The agents can be administered together or separately. In some embodiments both the proteolysis-enhancing agent(s) and the additional agent(s) are administered focally. In other embodiments the proteolysis-enhancing agent(s) are administered focally to the nervous system and the additonal agent(s) are administered by an alternate route (e.g., intravenously or orally).

X. Therapeutic Applications and Adiunct Therapy

The compositions and methods of the invention are of particular use in treating subjects who have experienced events such as stroke or injury (e.g., due to accident or surgery). The compositions and methods of the invention also find use for treating subjects suffering from a variety of other diseases and conditions including, but not limited to, neurodegenerative diseases such as multiple sclerosis, amyotrophic lateral sclerosis, subacute sclerosing panencephalitis, Parkinson's disease, Huntington's disease, muscular dystrophy, and conditions caused by nutrient deprivation or toxins (e.g., neurotoxins, drugs of abuse). Certain of the compositions and methods are of use for providing cognitive enhancement and/or for treating cognitive decline, e.g., “benign senescent forgetfulness”, “age-associated memory impairment”, “age-associated cognitive decline”, etc. (Petersen 2001; Bums 2002). These terms are intended to reflect the extremes associated with normal aging rather than a precursor to pathologic forms of memory impairment. Thus these conditions are distinct from Alzheimer's disease. Certain of the compositions and methods are of use for treating Alzheimer's disease. In certain embodiments of the invention the subject does not have, e.g., has not been diagnosed with, Alzheimer's disease. In certain embodiments of the invention the subject is not suspected of having Alzheimer's disease. In certain embodiments of the invention the subject has not been identified as having an increased risk for developing Alzheimer's disease. Methods for treating or preventing Alzheimer's disease, to the extent that any such methods are described and/or enabled in WO 01/58476 (PCT/EP01/01517) are explicitly excluded from certain embodiments of the instant invention.

Any of a wide variety of functional impairments may be treated using the compositions and methods of the invention. In one embodiment, the compositions are used to promote restoration of respiratory function after spinal cord injury (SCI). For this purpose the compositions are preferably administered to the spinal cord, e.g., intrathecally. If desired, administration can be localized to the region of the spinal cord injury, e.g., the cervical region of the spinal cord. Respiratory disorders are the leading cause of morbidity and mortality after SCI, affecting nearly half of all patients with a neurological deficit after SCI. Respiratory impairments resulting from cervical SCI, the most common clinical case, frequently render survivors chronically or permanently ventilator dependent, a sequelae which can dramatically compromise quality of life. There are no drug treatments for breathing disorders associated with SCI. Studies have established that the breathing system posseses a highly dynamic system of neuroplasticity which manifests both at the developmental stage as well as at the adulthood. Work in the laboratory of one of the inventors has demonstrated that even with nearly 50% phrenic respiratory motor region loss in the adult rat spinal cord, respiratory function can recover spontaneously in 5-6 weeks after a mid-cervical spinal cord injury. While the ultimate outcome from this neuroplasticity-mediated event is encouraging, the required lengthy period imposes serious life or death challenges to SCI patients. The present invention may significantly stimulate post-SCI respiratory neural circuit reorganization, and thus may quickly restore respiratory function after incomplete spinal cord transection, which is a frequent clinical occurrence.

Surgery for various conditions can sometimes result in damage to nerves. In another embodiment of the invention, the compositions and methods are used to regenerate, repair or otherwise restore function after nerves of the PNS supplying muscles, organs, or other parts of the body, or carrying information from a part of the body, have been necessarily or accidentally disconnected or damaged during surgery. In other embodiments, the present invention is used to regenerate, repair or prevent degeneration of nerves, e.g., nerves supplied by the spinal cord to the muscles, organs, or other parts of the body, or that enter the spinal cord from sensory receptors from the body. Other embodiments include regeneration or repair of damaged or degenerated nerves in the CNS, for example the optic nerve or the auditory nerve, or prevention of degeneration of axon tracts or fiber bundles in the CNS due to diseases, disorders or damage. These embodiments include, but are not limited to, the regrowth, recovery, repair or prevention of degeneration of ascending or descending fiber tracts and connections in the spinal cord, and of fiber tracts and connections in other structural and functional subdivisions of the CNS. Still other embodiments include rewiring or reorganizing brain pathways so as to elicit novel functions from existing brain regions. An example of this embodiment is enhancement of brain function, particularly when coupled with practice regimens that engage specific brain regions.

In certain embodiments of the invention the subject to whom a composition of the invention is administered is engaged in a program of rehabilitative therapy or training. Such programs typically ensue after injury or stroke, but also include programs of remediation and training in a variety of disorders of developmental or adult onset. Such programs are commonly employed in disorders such as dyslexia, autism, Asperger's Syndrome, Pervasive Developmental Disorders—Not Otherwise Specified, Tourette's Syndrome, Personality Disorders, Schizophrenia and related disorders. See, e.g., Diagnostic and Statistical Manual of Mental Disorders, 4th Ed. (DSM-IV) (American Psychiatric Association. (1994) Diagnostic and Statistical Manual (Am. Psychiatric Assoc., Washington, D.C.) for discussion of these disorders. Numerous rehabilitation programs for victims of stroke, spinal cord injury, and other forms of nervous system damage, are known to those skilled in the art, and the subject can be engaged in any such program. See, e.g., Gillen and Burkhardt, supra, for a discussion of suitable programs for victims of stroke. Similar programs may be used for victims of other forms of damage to the brain. See, e.g., Somers, supra, for a discussion of suitable programs for victims of spinal cord damage. Suitable programs for individuals suffering from damage to the PNS are also known in the art. A rehabilitation program is typically designed and recommended by a health care provider with knowledge in the area of rehabilitative therapy. The therapy sessions may involve the participation of a health care provider. However, the subject may also engage in sessions or tasks associated with the program without the assistance or supervision of the health care provider.

The subject can be engaged in the program in a defined temporal relation with respect to the administration of the agent. For example, the subject can be engaged in the program during a time period in which the agent is being administered and/or during which the agent is present in effective amounts in the nervous system. In some embodiments a dose of the agent is administered within a defined time period prior to engagement of the subject in a particular rehabilitative session or task. For example, the agent may be administered and/or may be present in an effective amount at any time up to 24 hours, 48 hours, or up to 1 week prior to the time at which the subject will be engaged in the session or task, or the agent may be administered and/or may be present in an effective amount at any time up to 24 hours, 48 hours, or up to 1 week following completion of the session or task. Typically the subject will be engaged in the program over a period of weeks, months, or years, i.e., the subject will participate in multiple therapy sessions over a period of time. The subject's participation in such sessions can be coordinated with administration of the agent so as to achieve an optimal effect. The beneficial effects of rehabilitative therapy may at least in part be due to structural and/or functional reorganization that occurs as a result of such therapy. Without wishing to be bound by any theory, the inventors propose that the proteolysis-enhancing activities of the agents disclosed herein may facilitate this process. Thus an at least additive and potentially synergistic effect may result.

The methods and compositions of the invention may be tested using any of a variety of animal models for injury to the nervous system. Models that may be used include, but are not limited to, rodent, rabbit, cat, dog, or primate models for thromboembolic stroke (Krueger and Busch, 2001; Gupta, 2004), models for spinal cord injury (Webb, et al., 2004), etc. See also Examples 6 and 7 and references in Schmidt and Leach, 2003. The methods and compositions may also be tested in humans.

A variety of different methods, including standardized tests and scoring systems, are available for assessing recovery of motor, sensory, behavioral, and/or cognitive function in animals and humans. Any suitable method can be used. As but one example, the American Spinal Injury Association score, which has become the principal instrument for measuring the recovery of sensory function in humans, could be used. See, e.g., Martinez-Arizala A. (2004), Thomas and Noga, (2004), Kesslak J P, Keirstead H S. (2003) for examples of various scoring systems and methods.

Preferred dose ranges for use in humans may be established by testing the agent(s) in tissue culture systems and in animal models taking into account the efficacy of the agent(s) and also any observed toxicity.

XI. Pharmaceutical Compositions

Suitable preparations, e.g., substantially pure preparations of the proteolysis-enhancing agents, optionally together with one or more additional active agents, may be combined with pharmaceutically acceptable carriers, diluents, solvents, etc., to produce an appropriate pharmaceutical composition. In general, methods and ingredients for producing a pharmaceutical composition known to one of skill in the art are used. The desription herein is for exemplary purposes and is not intended to be limiting. It is to be understood that the pharmaceutical compositions of the invention, when administered to a subject, are preferably administered for a time and in an amount sufficient to treat the disease or condition for whose treatment they are administered. Suitable modes of administration and formulations are described herein.

Further provided are pharmaceutically acceptable compositions comprising a pharmaceutically acceptable derivative (e.g., a prodrug) of any of the agents of the invention, by which is meant any non-toxic salt, ester, salt of an ester or other derivative of an agent of this invention that, upon administration to a recipient, is capable of providing, either directly or indirectly, an agent of this invention or an active metabolite or residue thereof. As used herein, the term “active metabolite or residue thereof” means that a metabolite or residue thereof also possesses similar activity to the parent agent. For example, rather than administering an active polypeptide, a zymogen (i.e., an inactive or less active enzyme pre-cursor that requires a biochemical change, such as a hydrolysis reaction revealing the active site, for it to become an active enzyme) could be administered.

The term “pharmaceutically acceptable carrier, adjuvant, or vehicle” refers to a non-toxic carrier, adjuvant, or vehicle that does not destroy the pharmacological activity of the agent with which it is formulated. Furthermore, it is recognized that preparation methods for the pharmaceutical compositions are preferably selected so as to not substantially reduce the activity of the agent with which they are formulated.

Pharmaceutically acceptable salts of certain of the agents of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates. Salts derived from appropriate bases include alkali metal (e.g., sodium and potassium), alkaline earth metal (e.g., magnesium), ammonium and N+(C1-4 alkyl) 4 salts. This invention also envisions the quatemization of any basic nitrogen-containing groups of the compounds disclosed herein. Water or oil-soluble or dispersible products may be obtained by such quatemization.

A pharmaceutical composition is formulated to be compatible with its intended route of administration. Pharmaceutical compositions suitable for injection or infusion typically include sterile aqueous solutions (where water soluble) or dispersions and sterile powders for the extemporaneous preparation of sterile injectable solutions or dispersion. Suitable carriers include physiological saline, bacteriostatic water, water for injection, dextrose solutions, phosphate buffered saline (PBS), or Ringer's solution. Antibacterial and/or antifungal agents, chelating agents such as ethylenediaminetetraacetic acid; buffers such as acetates, citrates or phosphates and agents for the adjustment of tonicity such as sodium chloride or dextrose can be included pH can be adjusted with acids or bases, such as hydrochloric acid or sodium hydroxide. It may be advantageous to formulate the compositions in dosage unit form for ease of administration and uniformity of dosage. Dosage unit form as used herein refers to physically discrete units suited as unitary dosages for the subject to be treated; each unit containing a predetermined quantity of active agent(s) calculated to produce the desired therapeutic effect in association with the required pharmaceutical carrier. The preparation can, for example, be enclosed in ampoules, disposable syringes or multiple dose vials made of glass or plastic.

Sterile injectable or infusable solutions can be prepared by incorporating the active compound in the required amount in an appropriate solvent, optionally with one or a combination of ingredients enumerated above, followed by filtered sterilization. Preferably solutions are free of endotoxin. Generally, dispersions are prepared by incorporating the active compound into a sterile vehicle which contains a basic dispersion medium and optionally other ingredients. In the case of sterile powders for the preparation of sterile solutions, the preferred methods of preparation are vacuum drying and freeze-drying (e.g., lyophilization) which yields a powder of the active ingredient plus any additional desired ingredient from a previously sterile-filtered solution thereof.

### EXAMPLES

### Example 1

### Monocular Deprivation Alters Spine Dynamics In Vivo

Materials and Methods

Monocular Deprivation.

Mice (C57/B16) expressing GFP (strain GFP-M) or YFP (strain YFP-H) in a subset of their cortical neurons (principally layer V pyramidal neurons) (Feng, G., et al., 2000) were anesthetized at postnatal days 26 or 40 and maintained in deep anesthesia using isoflurane. Monocular deprivation was performed by scoring the eyelids and then sealing them shut with tissue adhesive (Vetbond, 3M, St. Paul, Minn.). Mice were checked over the next 2-3 days to ensure that the eye remained closed. A total of 18 mice were used in the in vivo experiments (6 control, 4 deprived at p28; 4 control, 4 deprived at p42) and 24 mice were used in the slice experiments (15 control, 9 deprived).

Two photon imaging. Mice were prepared for in vivo imaging and imaged as described previously (Majewska and Sur, 2003). Briefly, primary visual cortex was identified using stereotaxic coordinates and either a small craniotomy was drilled in this area, or the skull was thinned without making a craniotomy. Imaging was performed with a custo m-made two-photon microscope consisting of a Ti:S laser (Tsunami, Spectra-physics, Menlo Park, Calif.) pumped by a 10W solid state source (Millennia, Spectra-physics) coupled to a modified Fluoview confocal scanhead (Olympus, Melville, N.Y.). Superficial dendrites were initially identified with wide-field epi-fluorescence illumination, and were subsequently imaged using a 60×, 0.9 N.A. lens with digital zoom (5-10×). Volumetric z-stacks were typically collected with a 800×600 acquisition window with 0.5-1 μm z-steps every five minutes over two hours. In some animals, at the end of the imaging session, injections of cholera toxin subunit B (CTB, List Biologic, Campbell, Calif.) coupled to Alexa Fluor 594 (Molecular Probes, Eugene, Oreg.) were made adjacent to imaged areas in order to facilitate identification of imaged cells after fixation. Mice were transcardially perfused and fixed with paraformaldehyde and coronal sections were cut to verify the location of imaged cells in VI.

Image Analysis.

Z-stack images were exported to MATLAB (Mathworks, Natick, Mass.) and processed using custom algorithms. Spine motility was analyzed on two-dimensional projections in ImageJ (NIH, Bethesda, Md.), where motility was defined as the average change in spine length per unit time (Lendvai et al., 2000; Majewska and Sur, 2003). Spine turnover was rarely observed in single imaging sessions. All values are presented as mean±standard error of the mean and all statistical analyses were performed using either a parametric t-test statistic for populations of monocularly deprived and control spines, or a parametric paired t-test statistic for enzymatic treatments.

Results

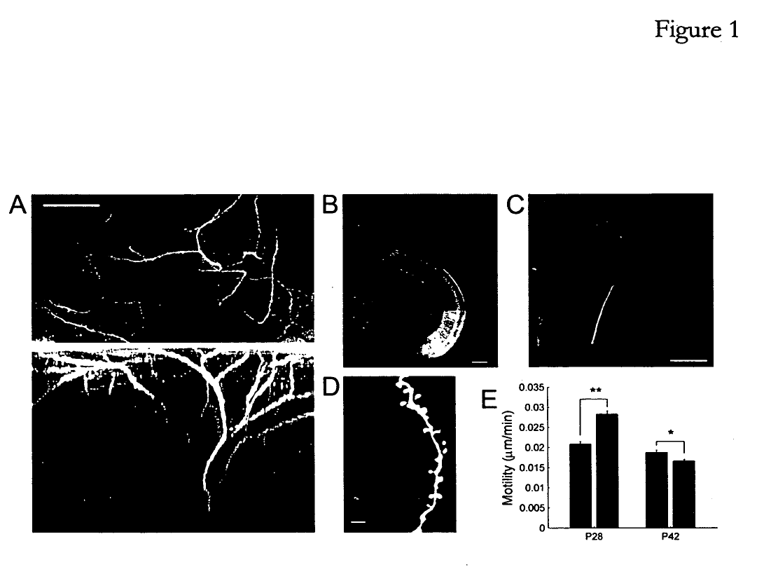

In order to examine whether monocular deprivation rapidly alters dendritic structure in a manner consistent with physiological changes, we visualized the dynamic structural properties of synapses by imaging dendritic spines in vivo. Spines from the apical arbor of layer V pyramidal neurons were imaged using two-photon microscopy (FIG. 1) at the height of the critical period (p28-29) (Gordon and Stryker, 1996) either with or without short-term monocular deprivation (two to three days, starting on p26). Spine motility in the binocular region of V1, contralateral to the deprived eye, was 35% higher than motility in control, non-deprived animals (FIG. 1E, n=147 control, 221 deprived, p<0.0001).

This result indicates that sensory deprivation in a fully innervated, yet plastic cortex was able to initiate a rapid sequence of events leading to increased structural dynamics at the level of individual spines. Such an increase in spine dynamics may reflect structural destabilization of a population of spines whose function is affected by visual deprivation. This, in turn, could precede a change in spine density. Interestingly, the upregulation of spine motility is restricted to the critical period as monocular deprivation at later ages (p42) had a modest effect of reducing spine motility (FIG. 1E, n=112 control, 153 deprived, p<0.005), suggesting that in older animals, deprivation may selectively stabilize spines. This may reflect alternate processes that regulate ocular dominance plasticity in the adult cortex (Sawtell et al., 2003).

Remodeling of the primary visual cortex during the critical period for ocular dominance plasticity is thought to progress from functional alterations in the response properties of single neurons (Gordon and Stryker, 1996; Hubel and Wiesel, 1970; Trachtenberg et al., 2000) to large anatomical shifts in axonal arborizations (Antonini et al., 1999; Antonini and Stryker, 1993; Shatz and Stryker, 1978). This idea is based solely on the relative timing of functional and anatomical events and further pre-supposes that the thalamocortical projection to layer IV is the principal indicator of altered connectivity following ocular dominance plasticity. However, recent evidence suggests that both functional (Trachtenberg et al., 2000) and anatomical (Trachtenberg and Stryker, 2001) changes in extragranular layers may precede, and subsequently inform, the altered connectivity in layer IV. Consistent with this hypothesis, we find that the dynamic properties of dendritic spines are substantially altered in laminar regions outside layer IV following two to three days of monocular deprivation during the peak of the critical period. Spines imaged both in vivo and in vitro showed elevated dynamics during this period, potentially reflecting a series of events that destabilized both synapses and spine structure. Longer periods of deprivation, greater than three days, may be required to alter the properties of spines in the middle region of the cortex, and to alter the pattern of thalamocortical connectivity (Antonini and Stryker, 1993).

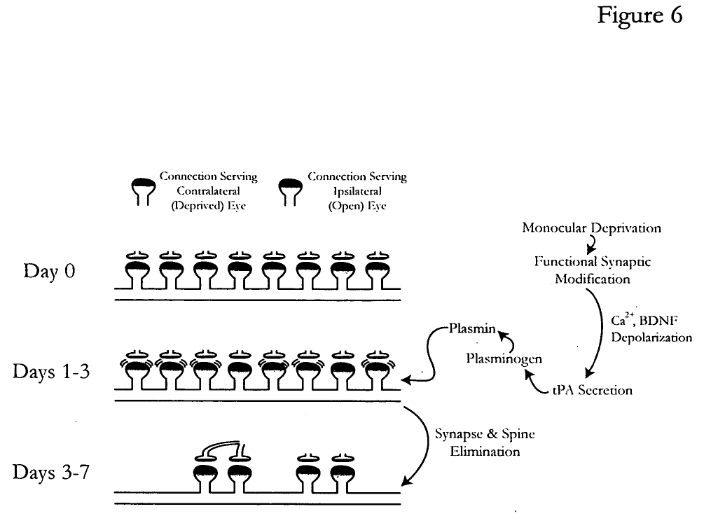

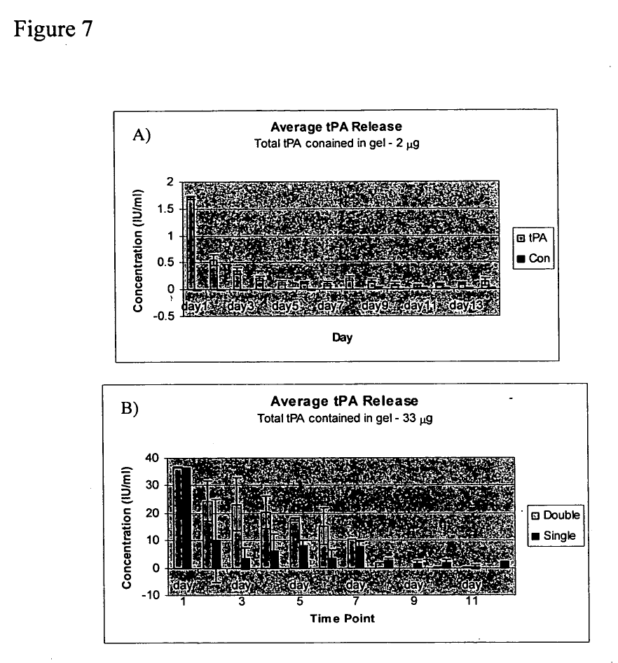

Processes such as long term synaptic depression, which can account for the rapid functional changes following monocular deprivation (Heynen et al., 2003), may also induce the translation from synaptic and functional modification to increased structural dynamics. Spines are likely to be influenced by persistent changes in synaptic efficacy as prolonged activation can induce the formation of new protrusions (Engert and Bonhoeffer, 1999; Maletic-Savatic et al., 1999) and accumulation of actin within dendritic spines (Fukazawa et al., 2003). Further, spines are stabilized by synaptic input (Fischer et al., 2000; McKinney et al., 1999) and significant loss of synaptic drive or long-lasting alteration of synaptic strength (Baranes et al., 1998) could lead to increased structural dynamics (Majewska and Sur, 2003). Monocular deprivation is likely to destabilize spines which are initially part of the neural circuitry driven by inputs from the deprived eye as synapses attempt to maintain homeostatic levels of activity by re-optimizing their connection to pre-synaptic partners or as a prelude to eventual synaptic loss and spine withdrawal (FIG. 6). With monocular deprivation, significant spine loss occurs within four days (Mataga, 2004), suggesting that synapses which had initially served the deprived eye are likely to have either been lost or have altered their connections within this short period of time.

### Example 2

### Monocular Deprivation Alters Spine Dynamics as Measured In Vitro

Materials and Methods

Monocular deprivation, imaging, and image analysis were performed as described in Example 1.

Slice preparation and enzyme application. Acute slices were prepared from p28-29 mice after deep anesthesia with sodium pentobarbitol (35 mg/kg, i.p.; Henry Schein Inc., Indianapolis, Ind.). The brain was removed and sectioned in cold (4° C.) solution containing (in mM): NaH₂PO₄ (1), NaHCO₃ (25), KCl (3), MgS0₄ (2), dextrose(10), sucrose (252), CaCl₂ (2.5), and kynurenic acid (5) in a coronal plane with a thickness of 300 μm. After sectioning, slices were transferred to a holding chamber containing room temperature artificial cerebrospinal fluid (ACSF) containing (in mM): NaH₂PO₄ (1), NaHCO₃ (25), KCl (3), MgS0₄ (2), dextrose (10), NaCl (126), and CaCl₂ (2.5). Slices were allowed to equilibrate for 1 hour before transferring to the microscopy submersion chamber which was continuously perfused with warm (35° C.) ACSF. For enzyme studies, slices were either perfused with 0.5 μg/mL tPA (American Diagnostica, Stamford, Conn.) for 45 minutes or were removed from the submersion chamber and incubated with 0.2 U/mL plasmin (Sigma, St. Louis, Mo.) in a small volume of ACSF for 45 minutes before being returned to the microscopy submersion chamber. Images were collected with two-photon microscopy every 6 minutes for 1.5 hours before and 1.5 hours after enzyme treatment.

Results

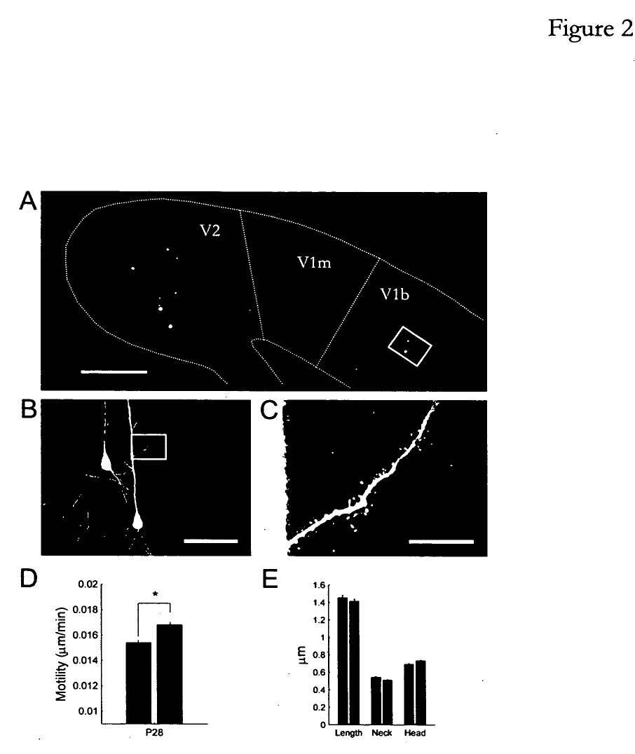

In vivo imaging with two-photon microscopy has the advantage of visualizing small structures in the living animal, though the signal to noise ratio becomes limiting as one images deeper into the tissue. To examine spines situated throughout the cortical layers, we took coronal slices from visual cortex from deprived and non-deprived animals (FIG. 2A). Spine motility in this preparation also decreases as development proceeds (Oray, in press), and reaches a stable level by the critical period. As with in vivo imaging, the motility of spines contralateral to the deprived eye was elevated (9%, n=503 control, 581 deprived, p<0.0001) following brief monocular deprivation as compared to non-deprived cortex (FIG. 2D). This change was not accompanied by a change either in average spine length, neck diameter, or head diameter (FIG. 2E). This suggests that an analysis of spine morphology in a fixed preparation could overlook an early indicator of potential structural remodeling which is only observed by examining the dynamic properties of spines.

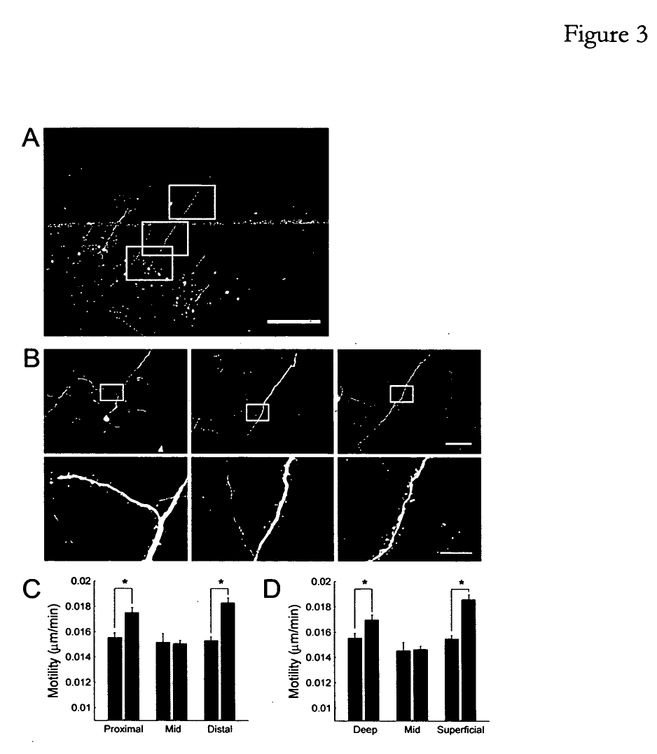

Motivated by the finding that electrophysiological changes in supra- and infragranular layers precede those in layer IV (Trachtenberg et al., 2000), we further divided our data set into multiple regions. These were defined as either proximal, middle, and distal based on their distance from the cell soma (FIG. 3C) or deep, middle, and superficial based on their distance from the cortical surface (FIG. 3D). With both of these analyses, the population of spines closest to the cortical surface, which are closest in laminar location to the population visualized in vivo, increased their motility ˜20% following deprivation as compared to non-deprived spines (distal, n=297 control, 231 deprived, p<0.0001; superficial n=260 control, 207 deprived, p<0.0001). Furthermore, an inspection of spines in other parts of the arbor reveals clear laminar differences in spine motility. Those spines located in the middle of the dendritic arbor, in layer IV, showed no increase in spine motility following deprivation (mid in FIG. 3C, n=42 control, 237 deprived, p>0.5; mid in FIG. 3D, n=64 control, 180 deprived, p>0.5), while those spines in the deep, infragranular region (proximal, n=164 control, 113 deprived, p<0.001; deep, 179 control, 194 deprived, p<0.005), showed an elevation of motility following deprivation. These results indicate that the distribution of synaptic contacts across the apical dendritic arbor are not homogenous, and that spines on a single neuron, separated only by several hundred micrometers, can experience differential environments for structural plasticity. Further, since the altered dynamics are present in the extragranular layers, they are likely to contribute to the remodeling of horizontal connections in these regions (Callaway and Katz, 1990; Callaway and Katz, 1991).

### Example 3

### ECM Degradation by tPA or Plasmin Alters Spine Dynamics

Materials and Methods

Monocular deprivation, slice preparation, imaging, and image analysis were performed as described in Examples 1 and 2.

Results

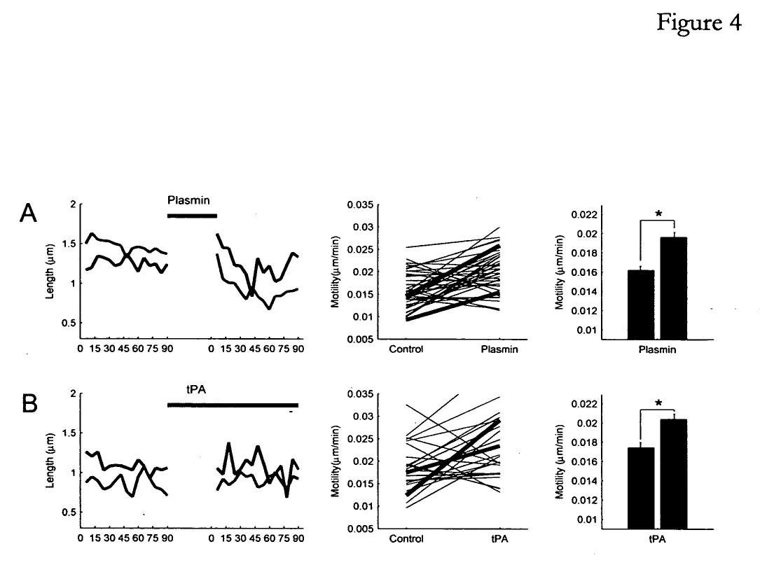

In order to examine the effect of tPA and plasmin on spine motility, spines from p28 animals were imaged in visual cortex slices before and after a 45 minute period of enzyme application. Treatment with either exogenous plasmin or exogenous tPA (without exogenous plasminogen) significantly increased spine motility (FIG. 4A, plasmin, 21% increase, n=191, p<0.0001; FIG. 4B, tPA, 17% increase, n=94, p<0.0001). There was no apparent laminar specificity to this effect, as spines situated through all layers of the cortex were equally effected by tPA and plasmin. These results indicate that proteolysis through the tPA/plasmin pathway can either induce structural plasticity or provide a permissive environment in which spine dynamics can be altered. While not wishing to be bound by any theory, since plasminogen knockout animals have impaired ocular dominance plasticity (Mataga, 2002), it is likely that tPA acts through plasmin to degrade the extracellular matrix, and that this may be one of the first steps in translating functional changes at the level of synapses into structural rearrangements.

### Example 4

### Monocular Deprivation Occludes Subsequent Effects of ECM Degradation in a Laminar Fashion

Materials and Methods

Monocular deprivation, slice preparation, imaging, and image analysis were performed as described in Examples 1 and 2.

Results

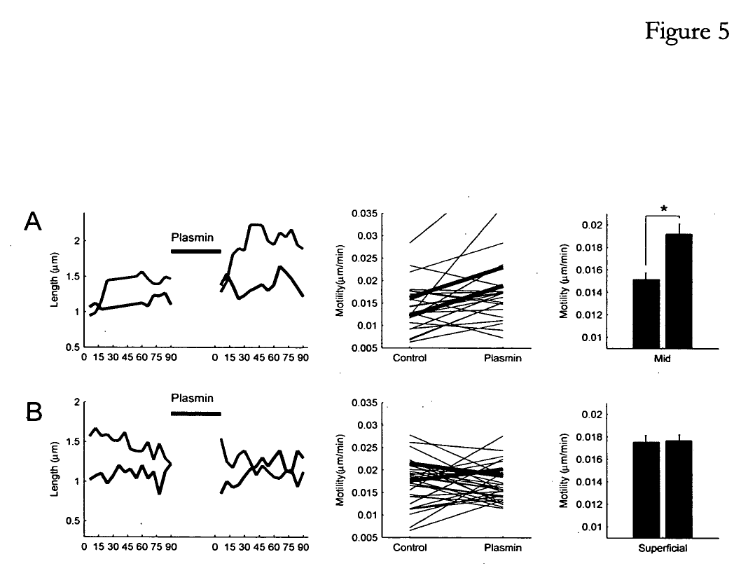

During ocular dominance plasticity, tPA exerts a critical role, as tPA knockout animals fail to enter a critical period (Mataga et al., 2002). To determine whether tPA/plasmin might be involved in the structural plasticity of dendritic spines during the critical period, we examined whether monocular deprivation would occlude a subsequent effect of exogenous tPA/plasmin. If a selective, local secretion of tPA is responsible for the laminar upregulation of spine motility, then those spines in the middle parts of the apical arbor, corresponding to layer IV, would be predicted to receive the least endogenous tPA, while extragranular spines should receive the most endogenous tPA. Consistent with this hypothesis, those regions where spine motility is upregulated by monocular deprivation (e.g. superficial layers) were unaffected by additional plasmin application (FIG. 5A, n=88, p>0.5), suggesting that monocular deprivation occluded a further increase in spine dynamics via proteolysis of the extracellular matrix. However, in middle regions where spine motility is unchanged following deprivation, spine motility was significantly increased by enzymatic treatment with plasmin (FIG. 5B, n=60, p<0.0001). These results strongly suggest that the tPA/plasmin proteolytic cascade is active in vivo following brief monocular deprivation in the infra- and supra-granular layers and provides a permissive extracellular environment in which spines are free to move, potentially as a prelude to changing their synaptic connectivity.

### Example 5

### Release of Enzymatically Active tPA from Hydrogel Discs

In order to demonstrate the release of tPA over time from a hydrogel matrix suitable for drug delivery, hydrogel discs containing various amounts of human recombinant tPA (Molecular Innovations, Inc., Southfield, Mich.) were fabricated and subjected to incubation in a PBS solution, during which release of tPA was measured over time.

The hydrogel consists of a poly(ethylene glycol) (PEG) core with poly(lactic acid) (PLA) linkages (i.e., it contains hPLA-b-PEG-PLA macromers) and has been previously described (Sawhney, et al., 1993; Burdick, et al., 2002). In order to fabricate the discs, the hydrogel macromer was combined with tPA (2 μg and 33 μg loading doses) and the photoinitiator 2-hydroxy-1-[4-(hydroxyethoxy)phenyl]-2-methyl-1-propanone, (Ciba-Geigy) in a PBS solution. The solution (50 μL) was placed into a mold of the desired dimensions and then crosslinked under UV light for 10 minutes to cause polymerization, thereby resulting in discs of hydrogel with dimensions of approximately 5 mm by 1 mm.

The hydrogel discs were placed in 0.5 ml of PBS solution and release was monitored over 14 days using an ELISA kit (Molecular Innovations) according to the manufacturer's directions. Three hydrogel discs were tested for each of the conditions (2 different loading doses each for single-chain and two-chain tPA), and the amount of tPA released was averaged at each time point. The ELISA kit measured only functionally active tPA.

We observed a linear relationship between tPA release and tPA loading dose, which allowed us to control the amount of tPA released by changing the amount of tPA initially loaded into the gel. The total amount of tPA released can be calculated from the concentrations and the fact that the discs were incubated in 0.5 ml PBS solution. For example, for two-chain tPA, on day 2 approximately 12.5 IU of tPA was released from the hydrogel. This information can be used to determine the amount of tPA and the amount of hydrogel needed to deliver a desired dose over time.

### Example 6

### Effect of tPA on Recoveryfrom Spinal Cord Injury

Materials and Methods

In a first set of experiments, 6 female Sprague-Dawley rats were anesthetized and spinal cord injury (SCI) was induced at TIO by using the New York University impactor with a 10 gm weight and a 12.5 mm weight drop. Behavioral tests were conducted on the first post-operative day and then weekly. The BBB (Basso, Beattie, Bresnahan) behavioral test was used to examine hind limb reflexes as well as coordinated use of the hind limbs (Basso et al., 1995; Basso, et al., 1996). This “BBB” scale has been adopted by the Multicenter Animal Spinal Cord Injury Study and by other workers in the field. Therefore, use of the BBB as an outcome measure after experimental SCI supports easier interlaboratory comparison of results.

Three days post-operatively, a second operation was conducted at T8-T9 for a bolus micro-injection of 10 μg of tPA (human two-chain tissue plasminogen activator; American Diagnostica, Inc.) reconstituted from lyophilized powder to 10 μg/10 μL) into three of the six rats. Following the bolus injection, an osmotic minipump (Alzet Model 2002: 14 day pump) (Durect Corp., Cupertino, Calif.) loaded with tPA (200 μL total volume, delivering 0.5 μL/hour, 10 μg tPA/day) was implanted at the side of injury and delivered tPA for 10 consecutive days. At the 6^{th} post-operative week, BDA and Fluorogold injections were made in cortex to assess the extent of corticospinal tract regrowth and reconnection, and at the 10^{th} post-operative week, animals were perfused and their spinal cords were removed for histological analysis. Implanted minipumps were saved for analysis of tPA activity in the remaining solution.

A second set of experiments was performed on a larger group of animals using the same techniques as the first except that Alzet Model 1007B:7 day pumps holding a total volume of 90 uL, infusing 0.5 uL/hour were used, and the tPA was delivered for 7 days rather than 10.

Results

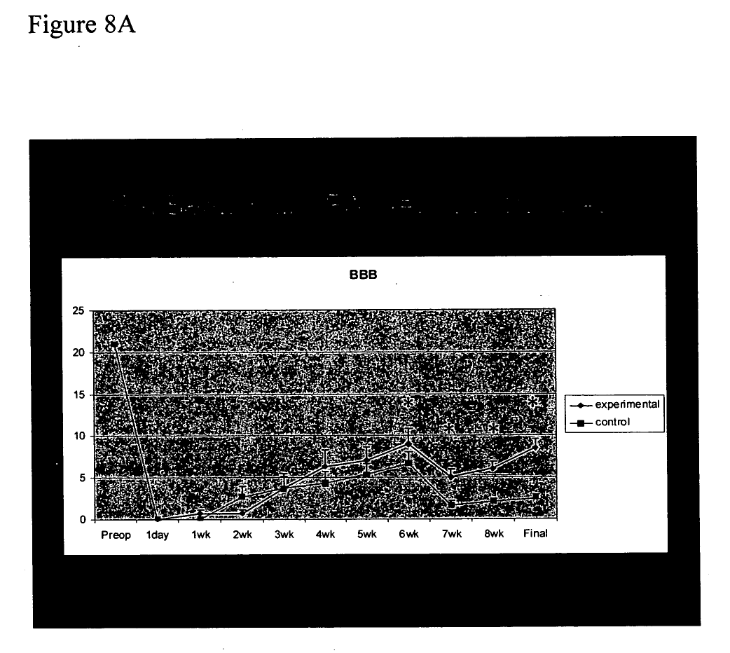

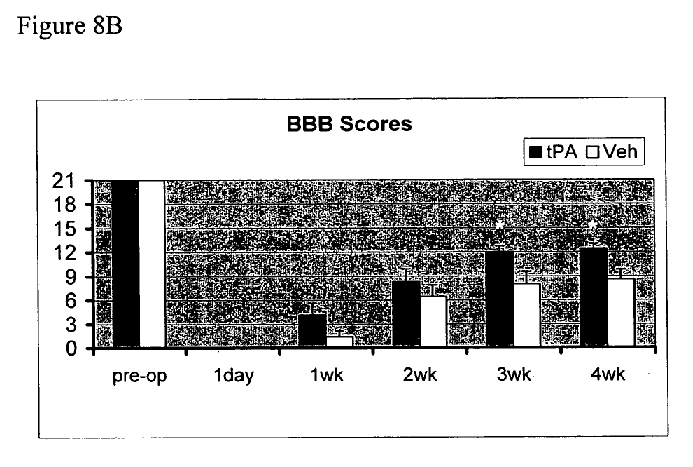

To examine the ability of tPA to promote recovery after injury, tPA was administered to animals who had been subjected to spinal cord injury. Functional and anatomical parameters were assessed. Pre-operatively, animals performed well on the BBB test, scoring a baseline value of 21. On the first post-operative day, all animals were significantly impaired on the BBB test, and their scores were reduced to 0. After 10 weeks of recovery, control animals achieved a final score of 2.5 on the BBB test while tPA-treated animals achieved a final score close to 9 (FIG. 8A). Although tPA treated animals did not recover to their baseline scores, the scores showed considerable recovery compared to control animals, up to the point where animals could bear weight on their hind legs and engage in locomotor activity. A BBB score of 9 is considered significant improvement. tPA-treated rats also showed improved BBB scores relative to control animals in a second experiment using more animals (FIG. 8B). Note that although the absolute values of the average BBB scores in the tPA-treated and untreated groups differ between the two experiments, in both cases there was a significant difference between the tPA-treated and control animals at the later time points.

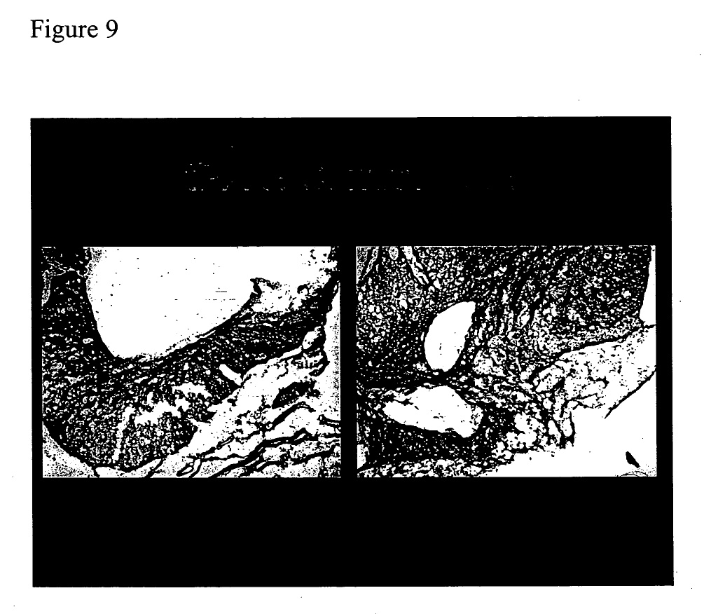

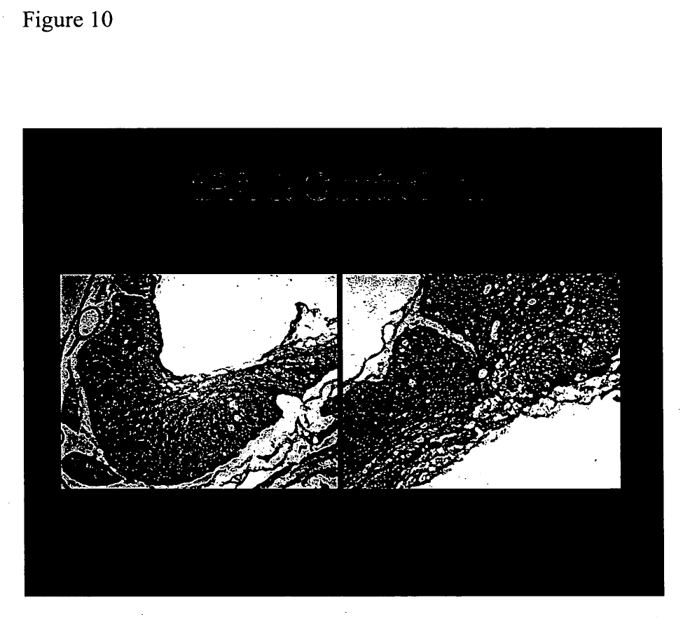

Anatomical analysis with hematoxylin and eosin staining suggests that the contusion site is remarkably clean after tPA in comparison to control (FIG. 9). In this figure, the dorsal spinal cord is to the right and lateral is to the bottom.

FIG. 10 presents micrographs showing the overall difference between rats receiving tPA or vehicle in the typical cross-sectioned area of residual total white matter (WM) at the injury epicenter (e.g. WM+hypomyelinated WM). Sections were stained with solvent blue [SB]/hematoxylin and eosin as described in Teng and Wrathall, 1997. tPA treatment significantly protected the integrity of the residual white matter, showing high quality myelin stain in the spared white matter which demonstrates existence of myelinated axons. In contrast, SB stain in the vehicle treated spinal cord was much weaker, indicating much lower quantity presence of myelin in the tissue. The vehicle-treated epicenter also showed large scale infiltration of non-neural tissue.

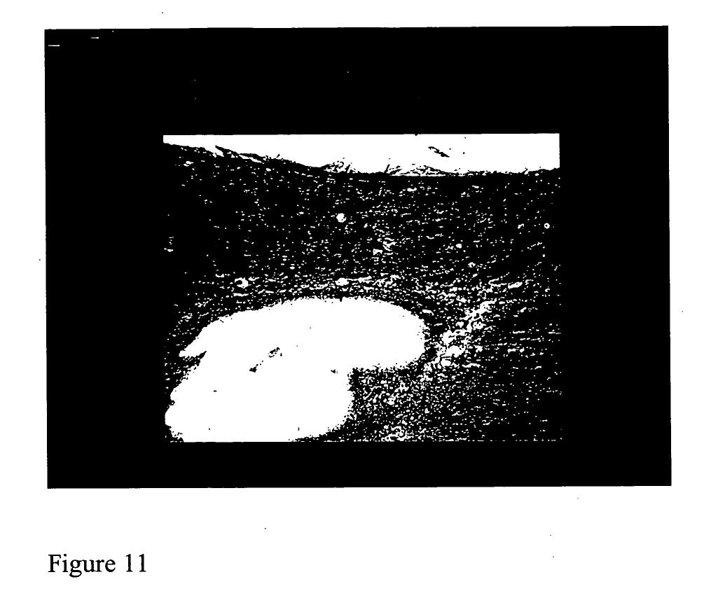

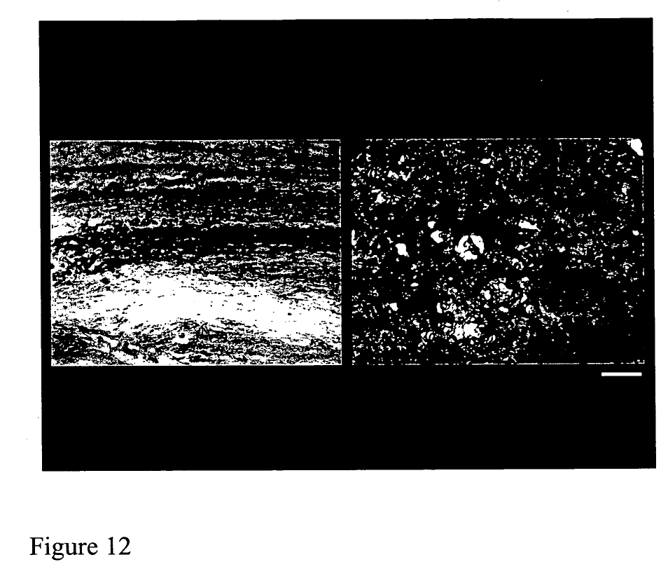

FIG. 11 shows a high magnification microscopic image of the spinal cord longitudinal profile of rats receiving tPA treatment in the typical area of residual total white matter (WM) at the injury epicenter (e.g. WM+hypomyelinated WM). Sections were stained with solvent blue [SB]/hematoxylin and eosin as described in Teng and Wrathall, 1997. High quality myelin stain is evident in the spared white matter which frequently showed longitudinally organized axonal arrays with healthy appearing myelin sheets (red arrows). The solvent blue staining is indicative of the clean cellular organization of the spinal cord after tPA treatment. The data suggests that tPA treatment may very likely have evoked neurite sprouting/regeneration and remyelination processes (see FIG. 12 for additional details).

BDA injections made in the cortex labeled axons which can be visualized in a longitudinal section passing through the impact site (FIG. 12, left). Likewise, sprouted axonal terminations close to the impact sight can be seen in a coronal section (FIG. 12, right).

Analysis of the excess, unused tPA from the experimental minipumps indicated that tPA maintains enzymatic activity for up to 10 days at rat body temperature when delivered from a minipump reservoir.

### Example 7

### Effect of IPA in an Animal Model of Stroke

Twenty rats were trained on a battery of behavioral tasks until they achieved an asymptotic level of competence. Rats then received occlusion of the middle cerebral artery (MCAO) according to standard procedures. After recovery from surgery, the rats were significantly impaired on all of the behavioral tasks. At the time of MCAO surgery, 10 of the 20 rats were also implanted with an osmotic mini-pump (Alzet model 2001, 7 day pump with 90 μL total volume and 1.0 μL/hour infusion) for intraventricular infusion contralateral to the site of the MCAO. The mini-pumps were filled with human two-chain tissue plasminogen activator (tPA; American Diagnostica, Inc.) at two different concentrations: 5 animals received 2 μg/day and 5 animals received 10 μg/day. tPA treatment was initiated 2 days following MCAO and was maintained for 7 days. Control and tPA-treated rats were subsequently tested weekly for behavioral recovery.

Initial results regarding one of the behavioral tests, forelimb inhibition, are striking. Normal animals rarely use their forelimbs while swimming across a test basin, a phenomenon referred to as “inhibition”. After stroke, this inhibition is reduced, so that forelimb use increases. With tPA treatment, animals used their forelimb much less than control animals. On other tests, there was less difference between tPA treated animals and controls though this and other data is still being analyzed.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. Furthermore, it is to be understood that the invention encompasses all variations, combinations, and permutations in which one or more limitations, elements, clauses, descriptive terms, etc., from one or more of the listed claims is introduced into another claim. In particular, any claim that is dependent on another claim can be modified to include one or more limitations found in any other claim that is dependent on the same base claim. In addition, it is to be understood that any particular embodiment of the present invention that falls within the prior art may be explicitly excluded from the claims. Since such embodiments are deemed to be known to one of ordinary skill in the art, they may be excluded even if not set forth explicitly herein. For example, any specific therapeutic agent, delivery vehicle, or condition to be treated can be excluded from one or more of the claims for any purpose. For example, Alzheimer's disease can be excluded from one or more of the claims. It is also to be understood that for any claim to a method for promoting recovery or reorganization in the nervous system of a subject, the invention includes a corresponding claim to a method of treating a subject that recites the same or similar steps (or steps appropriately modified according to the context of the claim).

### REFERENCES

Al-Anazi A, Bernstein M (2000). Modified stereotactic insertion of the Ommaya reservoir. J Neurosurg, 92:1050-1052.
Antonini, A., Fagiolini, M., and Stryker, M. P. (1999). Anatomical correlates of functional plasticity in mouse visual cortex. Journal of Neuroscience 19, 4388-4406.
Antonini, A., and Stryker, M. P. (1993). Rapid remodeling of axonal arbors in the visual cortex. Science 260, 1819-1821.
Asselbergs, et al., (1995) J. Biotechnol., 42(3):221-233.
Baranes, D., Lederfein, D., Huang, Y. Y., Chen, M., Bailey, C. H., and Kandel, E. R. (1998).
Tissue plasminogen activator contributes to the late phase of LTP and to synaptic growth in the hippocampal mossy fiber pathway. Neuron 21, 813-825.
Basso, D M, et al., (1995). A sensitive and reliable locomotor rating scale for open field testing in rats. J. Neurotrauma, 12(1):1-21.
Basso, D M., et al. (1996). Graded histological and locomotor outcomes after spinal cord contusion using the NYU weight-drop device versus transection. Exp. Neurol., 139(2): 244-256.
Benita et al. (1984) J. Pharm. Sci. 73:1721-1724.
Berry, M., et al., (2001) Gene therapy for central nervous system repair, Curr. Opin. Mol. Ther. 3: 338-49.
Biernaskie, J. and Corbett J. (2001) Enriched Rehabilitative Training Promotes Improved Forelimb Motor Function and Enhanced Dendritic Growth after Focal Ischemic Injury, The Journal of Neuroscience, 21(14):5272-5280.
Bizik, J., et al. (1990) Cell Regul.; 1(12): 895-905.
Blue, M. E., and Parnavelas, J. G. (1983). The formation and maturation of synapses in the visual cortex of the rat. II. Quantitative analysis. J Neurocytol 12, 697-712.
Bonhoeffer, T., and Yuste, R. (2002). Spine Motility: Phenomenology, Mechanisms, and Function. Neuron 35, 1019-1027.
Brody E N, Gold L. (2000) J Biotechnol., 74(1):5-13.
Brummelkamp, T. R., et al. (2002) A system for stable expression of short interfering RNAs in mammalian cells. Science 296:550-553.
Bunge, M B and Pearse, D D (2003) J Rehabil Res Dev. 40(4 Suppl 1):55-62.
Burns, A. & Zaudig, M (2002). Mild cognitive impairment in older people. The Lancet 360, 1963-1965.
Callaway, E. M., and Katz, L. C. (1990). Emergence and refinement of clustered horizontal connections in cat striate cortex. J Neurosci 10, 1134-1153.
Callaway, E. M., and Katz, L. C. (1991). Effects of binocular deprivation on the development of clustered horizontal connections in cat striate cortex. Proc Natl Acad Sci U S A 88, 745-749.
Chen, R., et al. (2002) Neuroscience, “Nervous System Reorganization Following Injury”, 111(4): 761-773.
Cho, IH, et a., (2004) Purification and characterization of six fibrinolytic serine-proteases from earthworm Lumbricus rubellus. J Biochem Mol Biol. 2004 Mar. 31;37(2):199-205.
Cotten and Birnstiel, (1989) EMBO J. 8:3861-3866.
Cramer, S., et al. (1997) A functional MRI study of subjects recovered from hemiparetic stroke, Stroke, 28: 2518-2527.
Dang W, Daviau T, Brem H (1996). Morphological characterization of polyanhydride biodegradable implant gliadel during in vitro and in vivo erosion using scanning electron microscopy. Pharm Res, 13:683:91.
De Felipe, J., Marco, P., Fairen, A., and Jones, E. G. (1997). Inhibitory synaptogenesis in mouse somatosensory cortex. Cereb Cortex 7, 619-634.
DeVivo, M. J., Epidemiology of traumatic spinal cord injury, in Kischblum, S., Campagnolo, D. I., DeLlisa, J. A. (eds.) Spinal Cord Medicine, 2002. Lippincott Williams & Wilkins, Philadelphia, pp. 69-81).
Dityatev, A., and Schachner, M. (2003). Extracellular matrix molecules and synaptic plasticity. Nat Rev Neurosci 4, 456-468.
Dunaevsky, A., Tashiro, A., Majewska, A., Mason, C., and Yuste, R. (1999). Developmental regulation of spine motility in the mammalian central nervous system. Proc Natl Acad Sci U S A 96, 13438-13443.
Elbashir, S M, et al., (2001) Duplexes of 21-nucleotide RNAs mediate RNA interference in cultured mammalian cells. Nature. 24;411(6836):494-8.
Elokdah H, et al. (2004) Tiplaxtinin, a novel, orally efficacious inhibitor of plasminogen activator inhibitor-1: design, synthesis, and preclinical characterization. J Med Chem. 47(14):3491-4.
Emptage, N., Bliss, T. V., and Fine, A. (1999). Single synaptic events evoke NMDA receptor-mediated release of calcium from internal stores in hippocampal dendritic spines. Neuron 22, 115-124.
Engert, F., and Bonhoeffer, T. (1999). Dendritic spine changes associated with hippocampal long-term synaptic plasticity. Nature 399, 66-70.
Fagiolini, M., Fritschy, J. M., Low, K., Mohler, H., Rudolph, U., and Hensch, T. K. (2004).
Specific GABAA circuits for visual cortical plasticity. Science 303, 1681-1683.
Fagiolini, M., and Hensch, T. K. (2000). Inhibitory threshold for critical-period activation in primary visual cortex. Nature 404, 183-186.
Fawcett, J W and Asher, R A (1999) The glial scar and central nervous system repair. Brain Res Bull. 49(6):377-91.
Feng, G., Mellor, R. H., Bernstein, M., Keller-Peck, C., Nguyen, Q. T., Wallace, M.,
Nerbonne, J. M., Lichtman, J. W., and Sanes, J. R. (2000). Imaging neuronal subsets in transgenic mice expressing multiple spectral variants of GFP. Neuron 28, 41-51.
Fischer, M., Kaech, S., Knutti, D., and Matus, A. (1998). Rapid actin-based plasticity in dendritic spines. Neuron 20, 847-854.
Fischer, M., Kaech, S., Wagner, U., Brinkhaus, H., and Matus, A. (2000). Glutamate receptors regulate actin-based plasticity in dendritic spines. Nat Neurosci 3, 887-894.
Fiumelli, H., Jabaudon, D., Magistretti, P. J., and Martin, J. L. (1999). BDNF stimulates expression, activity and release of tissue-type plasminogen activator in mouse cortical neurons. Eur J Neurosci 11, 1639-1646.
Fleming A B, Saltzman W M (2002). Pharmacokinetics of the carmustine implant. Clin Pharmackinet, 41:403-19.
Fukazawa, Y., Saitoh, Y., Ozawa, F., Ohta, Y., Mizuno, K., and Inokuchi, K. (2003).
Hippocampal LTP Is Accompanied by Enhanced F-Actin Content within the Dendritic Spine that Is Essential for Late LTP Maintenance In Vivo. Neuron 38, 447-460.
Furlan, M., et al., (1996) Spontaneous neurological recovery after stroke and the fate of the ischemic penumbra”, Ann. Neurol., 40:216-226.
Gale K, Kerasidis H, Wrathall J R (1985) Spinal cord contusion in the rat: behavioral analysis of functional neurological impairment. Exp. Neurol 88:123-134.
Galicich J H, Guido L J (1974). Ommaya device in carcinomatous and leukemic meningitis.
Surgical experience in 45 cases. Surg Clin North Am 54:915-922.
Ge, T., et al., (2005) Cloning of thrombolytic enzyme (lumbrokinase) from earthworm and its expression in the yeast Pichia pastoris. Protein Expr Purif. 2005 July;42(1):20-8.
Gils, A., et al. (2002) Characterization and comparative evaluation of a novel PAI-1 inhibitor. Thromb Haemost. 88(1):137-43.
Goldman S. (2005) Stem and progenitor cell-based therapy of the human central nervous system. Nat Biotechnol.23(7):862-71.
Gordon, J. A., and Stryker, M. P. (1996). Experience-dependent plasticity of binocular responses in the primary visual cortex of the mouse. Journal of Neuroscience 16, 3274-3286.
Gray, E. (1959). Electron microscopy of synaptic contacts on dendritic spines of the cerebral cortex. Nature 183, 1592-1593.
Gualandris, A., Jones, T. E., Strickland, S., and Tsirka, S. E. (1996). Membrane depolarization induces calcium-dependent secretion of tissue plasminogen activator. J Neurosci 16, 2220-2225.
Guo, J T, et al., Nucleic Acids Res. 32 (Web Server issue):W522-5, Jul. 1, 2004).
Gupta, Y K and Briyal, S., (2004) Animal models of cerebral ischemia for evaluation of drugslndian J Physiol Pharmacol. 48(4):379-94.
Hall, A. (1998). Rho GTPases and the actin cytoskeleton. Science 279, 509-514.
Han, S. -O., R. I. Mahato, Y. K. Sung, and S. W. Kim. (2000) Development of Biomaterials for gene therapy. Mol. Therapy 2:302-317.
Harenberg, (1998), Med. Res. Rev., 18:1-20.
Heinemann U., et al., (2001); Curr Opin Biotechnol. 12(4):348-54.
Hennan J K (2005) Evaluation of PAI-039 [{1-Benzyl-5-[4-(trifluoromethoxy)phenyl]-1H-indol-3-yl}(oxo)acetic Acid], a Novel Plasminogen Activator Inhibitor-1 Inhibitor, in a Canine Model of Coronary Artery Thrombosis. Pharmacol Exp Ther. 314(2):710-6.
Hensch, T. K., Fagiolini, M., Mataga, N., Stryker, M. P., Baekkeskov, S., and Kash, S. F. (1998). Local GABA circuit control of experience-dependent plasticity in developing visual cortex. Science 282, 1504-1508.
Hering, H., and Sheng, M. (2001). Dendritic spines: structure, dynamics and regulation. Nat Rev Neurosci 2, 880-888.
Heynen, A. J., Yoon, B. J., Liu, C. H., Chung, H. J., Huganir, R. L., and Bear, M. F. (2003).
Molecular mechanism for loss of visual cortical responsiveness following brief monocular deprivation. Nat Neurosci 6, 854-862.
Higgins D L and Bennett W F, (1990) Tissue Plasminogen Activator: The Biochemistry and Pharmacology of Variants Produced by Mutagenesis. Annual Review of Pharmacology and Toxicology Vol. 30: 91-121.
Huang, Z. J., Kirkwood, A., Pizzorusso, T., Porciatti, V., Morales, B., Bear, M. F., Maffei, L., and Tonegawa, S. (1999). BDNF regulates the maturation of inhibition and the critical period of plasticity in mouse visual cortex. Cell 98, 739-755.
Hubel, D. H., and Wiesel, T. N. (1970). The period of susceptibility to the physiological effects of unilateral eye closure in kittens. J Physiol 206, 419-436.
Johannson, B. (2000) “Brain Plasticity and Stroke Rehabilitation”, Stroke, 31:223-230.
Kanematsu, A., et al. (2004) Collagenous matrices as release carriers of exogenous growth factors. Biomaterials. 25(18):4513-20.
Kesslak J P, Keirstead H S. (2003) Assessment of behavior in animal models of spinal cord injury. J Spinal Cord Med. 26(4):323-8.
Koester, H. J., and Sakmann, B. (1998). Calcium dynamics in single spines during coincident pre- and postsynaptic activity depend on relative timing of back-propagating action potentials and subthreshold excitatory postsynaptic potentials. Proc Natl Acad Sci USA 95, 9596-9601.
Krueger K, Busch E. Protocol of a thromboembolic stroke model in the rat: review of the experimental procedure and comparison of models. Invest Radiol. 2002. 37(11):600-8.
Krystosek, A., and Seeds, N. W. (1981). Plasminogen activator release at the neuronal growth cone. Science 213, 1532-1534.
Lamer T J (1994). Treatment of cancer-related pain: when orally administered medications fail. Mayo Clin Proc, 69:473-80.
Laske, D W, et al., 1997 Nat. Med. Tumor regression with regional distribution of the targeted toxin TF-CRM107 in patients with malignant brain tumors.3(12):1362-8.
Leamey C A, et al., (2001) Disruption of retinogeniculate pattern formation by inhibition of soluble guanylyl cyclase. J Neurosci. 21(11):3871-80.
Lendvai, B., Stern, E. A., Chen, B., and Svoboda, K. (2000). Experience-dependent plasticity of dendritic spines in the developing rat barrel cortex in vivo. Nature 404, 876-881.
Liang, A., et al., (2005) Characterization of a small molecule PAI-1 inhibitor, ZK4044. Thromb Res. 115(4):341-50. Epub 2004 Nov. 13.
Liberatore G T, et al., Vampire bat salivary plasminogen activator (desmoteplase): a unique fibrinolytic enzyme that does not promote neurodegeneration. Stroke. 2003 February; 34(2):537-43.
Liepert, J., et al. (2000) Treatment-Induced Cortical Reorganization After Stroke in Humans, Stroke, 31:1210-1216.
Machado M, Salcman M, Kaplan R S, Montgomery E (1985). Expanded role of the cerebrospinal fluid reservoir in neuroongocology: indications, causes of revision, and complications. Neurosurgery 17:600-603.
Majewska, A., Brown, E., Ross, J., and Yuste, R. (2000a). Mechanisms of calcium decay kinetics in hippocampal spines: role of spine calcium pumps and calcium diffusion through the spine neck in biochemical compartmentalization. J Neurosci 20, 1722-1734.
Majewska, A., and Sur, M. (2003). Motility of dendritic spines in visual cortex in vivo: Changes during the critical period and effects of visual deprivation. Proc Natl Acad Sci U S A 100, 16024-16029.
Majewska, A., Tashiro, A., and Yuste, R. (2000b). Regulation of spine calcium dynamics by rapid spine motility. J Neurosci 20, 8262-8268.
Maletic-Savatic, M., Malinow, R., and Svoboda, K. (1999). Rapid dendritic morphogenesis in CA1 hippocampal dendrites induced by synaptic activity. Science 283, 1923-1927.
Martinez-Arizala A. (2003) Methods to measure sensory function in humans versus animals. J Rehabil Res Dev. 40(4 Suppl 1):35-9.
Mataga N, Mizuguchi Y, Hensch T K (2004) Experience-dependent pruning of dendritic spines in visual cortex by tissue plasminogen activator. Neuron 44:1031-1041.
Mataga, N., Nagai, N., and Hensch, T. K. (2002). Permissive proteolytic activity for visual cortical plasticity. Proc Natl Acad Sci U S A 99, 7717-7721.
Matus, A., Ackermann, M., Pehling, G., Byers, H. R., and Fujiwara, K. (1982). High actin concentrations in brain dendritic spines and postsynaptic densities. Proc Natl Acad Sci U S A 79, 7590-7594.
Mathiowitz and Langer (1987) J. Controlled Release 5:13-22.
Mathiowitz et al. (1987) Reactive Polymers 6:275-283.
Mathiowitz et al. (1988) J. Appl. Polymer Sci. 35:755-774.
Mathiowitz et al. (1990) Scanning Microscopy 4:329-340;
Mathiowitz et al. (1992) J. Appl. Polymer Sci., 45:125-134.
McKinney, R. A., Capogna, M., Dürr, R., and Gahwiler, B. H. (1999). Miniature synaptic events maintain dendritic spines via AMPA receptor activation. Nature Neuroscience 2, 44-49.
McManus, M. T., and P. A. Sharp. (2002) Gene silencing in mammals by short interfering RNAs. Nature Rev. Genetics. 3:737-747.
Milwidsky, et al. (1991), Thrombo. Haemostat., 65:389-393.
Muller, C. M., and Griesinger, C. B. (1998). Tissue plasminogen activator mediates reverse occlusion plasticity in visual cortex. Nat Neurosci 1, 47-53.
Nelles, G., et al. (1999) “Reorganization of sensory and motor systems in hemiplegic stroke patients. A positron emission study.”, Stroke 30:1510-1516.
Nishiyama M, et al., (2003) Cyclic AMP/GMP-dependent modulation of Ca2+ channels sets the polarity of nerve growth-cone turning. Nature. 423(6943):990-5.
Noble L J, Wrathall J R (1985) Spinal cord contusion in the rat: morphometric analyses of alterations in the spinal cord. Exp Neurol 88:135-149.
Noble L J, Wrathall J R (1989a) Correlative analysis of lesion development and functional status after graded spinal cord contusive injuries in the rat. Exp Neurol 103:34-40.
Noble L J, Wrathall J R (1989b) Distribution and time course of protein extravasation in the spinal cord after contusive injury. Brain Res 482:57-66.
Obbens EAMT, Leavents M E, Beal J W, Lee Y Y (1985). Ommaya reservoirs in 387 cancer patients: a 15-year experience. Neurology 35:1274-1278.
Ohtani A, Inhibitory effect of a new butadiene derivative on the production of plasminogen activator inhibitor-1 in cultured bovine endothelial cells.
J Biochem (Tokyo). 1996 December; 120(6):1203-8. Related Articles, Links
Olson, C. R., and Freeman, R. D. (1975). Progressive changes in kitten striate cortex during monocular vision. J Neurophysiol 38, 26-32.
Ommaya A K, Punjab M B (1963). Subcutaneous reservoir and pump for sterile access to ventricular cerebrospinal fluid. Lancet, 2:983-984.
Oray S, Majewska A, Sur M (in press) Effects of synaptic activity on dendritic spine motility of developing cortical layer 5 pyramidal neurons. Cerebral Cortex.
Paice J A, Penn R D, Shott S (1996). Intraspinal morphine for chronic pain: a retrospective, multicenter study. J Pain Symptom Manage, 11:71-80.
Panjabi M, Wrathall J R (1988) Biomechanical analysis of spinal cord injury and functional loss. Spine 13:1365-1370.
Parkinnen (1993), J. Biol. Chem. 268: 19726-19738.
Petersen, R. C., et al., (2001). Current Concepts in Mild Cognitive Impairment. Arch. Neurol. 58, 1985-1992.
Pizzorusso, T., Medini, P., Berardi, N., Chierzi, S., W. Fawcett, J., and Maffei, L. (2002). Reactivation of ocular dominance plasticity in the adult visual cortex. Science 298, 1248-1251.
Qian, Z., Gilbert, M. E., Colicos, M. A., Kandel, E. R., and Kuhl, D. (1993). Tissue-plasminogen activator is induced as an immediate-early gene during seizure, kindling and long-term potentiation. Nature 361, 453-457.
Raines A, Dretchen K L, Marx K, Wrathall J R (1988) Spinal cord contusion in the rat: somatosensory evoked potentials as a function of graded injury. J Neurotrauma 5:151-160.
Ramos B P, et al., Dysregulation of protein kinase a signaling in the aged prefrontal cortex: new strategy for treating age-related cognitive decline. Neuron, 40(4):835-45.
Rijken, D. C. and Collen, D. (1981) Purification and characterization of the plasminogen activator secreted by human melanoma cells in culture. J. Biol. Chem., 256, 7035-7042.
Roberts L J, Finch P M, Goucke C R, Price L M (2001). Outcome of intrathecal opioids in chronic non-cancer pain. Eur J Pain, 5:353-61.
Sakata, et al. (1999), Am. Heart J., 137:1094-1099.
Sali, A. and Blundell, T L, (1993) J. Mol. Biol., 234, 779-815.
Santini, J T, et al. (2000) Microchips as Controlled Drug-delivery Devices Angewandte Chemie, International Edition, Vol. 39, pp. 2396-2407.
Sawtell, N. B., Frenkel, M. Y., Philpot, B. D., Nakazawa, K., Tonegawa, S., and Bear, M. F. (2003). NMDA Receptor-Dependent Ocular Dominance Plasticity in Adult Visual Cortex. Neuron 38, 977-985.
Schlaug, G., et al. (1999) The ischemic penumbra: operationally defined by diffusion and perfusion MRI. Neurology. 53(7):1528-37.
Schlott, et al. (1997), J. Biol. Chem. 272: 6067-6072.
Schmidt, C. E. and Leach, J. B., Neural tissue engineering: strategies for repair and regeneration. Annu. Rev. Biomed. Eng., 2003.5:293-347.
Shatz, C. J., and Stryker, M. P. (1978). Ocular dominance in layer IV of the cat's visual cortex and the effects of monocular deprivation. J Physiol 281, 267-283.
Siconolfi, L. B., and Seeds, N. W. (2001). Induction of the plasminogen activator system accompanies peripheral nerve regeneration after sciatic nerve crush. J Neurosci 21, 4336-4347.
Sprengers, E. D. and Kluft, C. (1987). Plasminogen activator inhibitors. Blood 69, 381-387.
Star, E. N., Kwiatkowski, D. J., and Murthy, V. N. (2002). Rapid turnover of actin in dendritic spines and its regulation by activity. Nat Neurosci 5, 239-246.
Sun, et al., (2000) Pharmacol. Rev., 52:325.
Takenaga, M., et al., (2004) Optimum formulation for sustained-release insulin. Int J Pharm. 271(1-2):85-94.
Teng Y D, Wrathall J R (1996) Evaluation of cardiorespiratory parameters in rats after spinal cord trauma and treatment with NBQX, an antagonist of excitatory amino acid receptors. Neurosci Lett 209:5-8.
Teng, Y D and Wrathall, J R (1997) J. Neuroscience, 17(11), pp. 4359-4366.
Teng, Y. D., et al. (2002) Functional recovery following traumatic spinal cord injury mediated by a unique polymer scaffold seeded with neural stem cells. Proc Natl Acad Sci U S A, 99(5): p. 3024-9.
Teng, Y. D., et al. (2004). Proc. Natl. Acad. Sci. 101(9), pp. 3071-3076.
Thomas C K, Noga B R. (2003) Physiological methods to measure motor function in humans and animals with spinal cord injury. J Rehabil Res Dev. 40(4 Suppl 1):25-33.
Thomas, N., and Klibanov, A. M. (2003) Non-viral gene therapy: polycation-mediated DNA delivery. Appl. Microbiol. Biotechnol. 62:27-34.
Toombs C F. (2001) Alfimeprase: pharmacology of a novel fibrinolytic metalloproteinase for thrombolysis. Haemostasis. 31(3-6): 141-7.
Trachtenberg, J. T., and Stryker, M. P. (2001). Rapid anatomical plasticity of horizontal connections in the developing visual cortex. J Neurosci 21, 3476-3482.
Trachtenberg, J. T., Trepel, C., and Stryker, M. P. (2000). Rapid extragranular plasticity in the absence of thalamocortical plasticity in the developing primary visual cortex. Science 287, 2029-2032.
Tutak U, Doleys D M (1996). Intrathecal infusion systems for treatment of chronic low back and leg pain of noncancer origin. South Med J, 89:295-300.
Usman, et al., (1996) Curr. Opin. Struct. Biol., 1:527.
Webb, A A, et al. (2004) Behavioural analysis of the efficacy of treatments for injuries to the spinal cord in animals. Vet Rec. 155(8):225-30.
Werb, Z. (1997). ECM and cell surface proteolysis: regulating cellular ecology. Cell 91, 439-442.
Westphal M, Hild D C, Bortey E, Delavault P, Olivares R, Warnke P C, Whittle I R, Jaaskelainen J, Ram Z (2003). A phase 3 trial of local chemotherapy with biodegradable carmustine (BCNU) wafers (Gliadel wafers) in patients with primary malignant glioma. Neuro-oncol 5:79-88.
White (1998), J. Am. Coll. Cardiol., 31: 487-496.
Wiesel, T. N., and Hubel, D. H. (1963). Single-Cell Responses in Striate Cortex of Kittens Deprived of Vision in One Eye. J Neurophysiol 26, 1003-1017.
Winkemuller M, Winkemuller W (1996). Long-term effects of continuous intrathecal opioid treatment in chronic pain of nonmalignant etiology. J Neurosurg, 85:458-467.
Wishart D., (2005) Curr Pharm Biotechnol., 6(2):105-20.
Wrathall J R, Pettegrew R, Harvey F (1985) Spinal cord contusion in the rat: production of graded, reproducible injury groups. Exp Neurol 88:108-122.
Wu M P, Tamada J A, Brem H, Langer R (1994). In vivo versus in vitro degradation of controlled release polymers for intracranial surgical therapy. J Biomed Mater Res, 28:387-95.
Xerri, C. et al. (1998) Plasticity of primary somatosensory motor cortex paralleling sensorimotor skill recovery from stroke in adult monkeys, J. Neurophysiol., 79:2119-2148.
Ye, B., et al, (2004) Synthesis and biological evaluation of piperazine-based derivatives as inhibitors of plasminogen activator inhibitor-1 (PAI-1). Bioorg Med Chem Lett. 14(3):761-5.
Yelverton, E., et al., (1983) Cloning and expression of human tissue-type plasminogen activator cDNA in E. coli., Nature, 301(5897):214-21
Yuste, R., and Denk, W. (1995). Dendritic spines as basic functional units of neuronal integration. Nature 375, 682-684.
Zavalova, L., (1996) Genes from the medicinal leech (Hirudo medicinalis) coding for unusual enzymes that specifically cleave endo-epsilon (gamma-Glu)-Lys isopeptide bonds and help to dissolve blood clots. Mol Gen Genet. 253(1-2):20-5.
Zavalova L, et al., (2002) Fibrinogen-fibrin system regulators from bloodsuckers. Biochemistry (Mosc). 67(1):135-42.

We claim:

1. A method for promoting recovery or reorganization in the nervous system of a subject comprising the step of:
focally administering a composition comprising a proteolysis-enhancing agent to the central or peripheral nervous system of a subject in need of enhancement of recovery or reorganization of the nervous system as a result of ischemic, hemorrhagic, neoplastic, degenerative, or traumatic damage to the nervous system.

2. The method of claim 1, wherein the proteolysis-enhancing agent is a protease.

3. The method of claim 1, wherein the proteolysis-enhancing agent is plasmin, a plasminogen activator, or an inhibitor of an endogenous plasminogen activator inhibitor.

4. The method of claim 1, wherein the proteolysis-enhancing agent is tissue plasminogen activator (tPA).

5. The method of claim 1, wherein the proteolysis-enhancing agent is plasmin.

6. The method of claim 1, wherein the proteolysis-enhancing agent promotes degradation of a component of the extracellular matrix.

7. The method of claim 1, wherein the proteolysis-enhancing agent directly or indirectly degrades fibrin.

8. The method of claim 1, wherein focal administration is performed between 1 day and 1 month following a specific damaging event.

9. The method of claim 1, wherein focal administration is performed between 1 week and 1 month following a specific damaging event.

10. The method of claim 1, wherein focal administration is performed at least 1 month following a specific damaging event.

11. The method of claim 1, wherein focal administration is performed after the subject has reached a plateau of functional recovery following a specific damaging event.

12. The method of claim 1, wherein the damage involves the central nervous system.

13. The method of claim 12, wherein the damage involves the brain.

14. The method of claim 12, wherein the damage involves the spinal cord.

15. The method of claim 1, wherein the damage involves the peripheral nervous system.

16. The method of claim 1, wherein focal administration is achieved by implanting into the subject a drug delivery device that releases the proteolysis-enhancing agent over a period of time at or in the vicinity of a desired location in the central or peripheral nervous system.

17. The method of claim 16, wherein the desired location is an area of ischemic, hemorrhagic, neoplastic, degenerative, or traumatic damage in the central or peripheral nervous system, or is an oppositely located brain hemisphere.

18. The method of claim 16, wherein the drug delivery device comprises a pump.

19. The method of claim 16, wherein the drug delivery device comprises a biocompatible polymer.

20. The method of claim 19, wherein the polymer is biodegradable.

21. The method of claim 1, wherein the composition comprises a plurality of polymeric microparticles or nanoparticles having the proteolysis-enhancing agent associated therewith.

22. The method of claim 1, wherein the proteolysis-enhancing agent is delivered in a solution that forms a gel following contact with physiological fluids.

23. The method of claim 1, wherein the proteolysis-enhancing agent is delivered in an amount effective to promote structural reorganization of synaptic connections, increase formation of new synaptic connections, increase dendritic spine motility, promote growth of axons, or any combination of the foregoing.

24. The method of claim 1, wherein the proteolysis-enhancing agent is delivered in an amount effective to promote growth or sprouting of axons, promote structural reorganization of synaptic connections, increase formation of new synaptic connections, increase dendritic spine motility, or any combination of the foregoing over a period of at least 1 week.

25. The method of claim 1, wherein the proteolysis-enhancing agent is delivered in an amount effective to promote growth or sprouting of axons, promote structural reorganization of synaptic connections, increase formation of new synaptic connections, increase dendritic spine motility, or any combination of the foregoing over a period of at least 1 month.

26. The method of claim 1, wherein the proteolysis-enhancing agent is delivered in an amount effective to promote growth or sprouting of axons, promote structural reorganization of synaptic connections, increase formation of new synaptic connections, increase dendritic spine motility, or any combination of the foregoing over a period of at least 3 months.

27. The method of claim 1, wherein the proteolysis-enhancing agent is delivered in an amount effective to promote growth or sprouting of axons, promote structural reorganization of synaptic connections, increase formation of new synaptic connections, increase dendritic spine motility, or any combination of the foregoing over a period of at least 1 year.

28. The method of claim 1, wherein the composition is administered by injecting or infusing it at or in the vicinity of a desired location in the central or peripheral nervous system.

29. The method of claim 28, wherein the desired location is an area of ischemic, hemorrhagic, neoplastic, degenerative, or traumatic damage in the central or peripheral nervous system, or is an oppositely located brain hemisphere.

30. The method of claim 1, wherein the composition is administered intrathecally.

31. The method of claim 1, wherein the composition further comprises an agent selected from the group consisting of neural growth enhancing agents, which are optionally selected from among neurotransmitters or analogs thereof, neurally active growth factors, neural signaling molecules, neurally active small molecules, and neurally active metals.

32. The method of claim 1, further comprising the step of:
engaging the subject in a program of rehabilitation designed to promote functional recovery following ischemic, hemorrhagic, neoplastic, or traumatic damage to the nervous system, wherein the subject is so engaged during at least part of the time interval during which the agent is administered or during which the agent remains active in the nervous system of the subject.

33. A method of treating a subject in need of enhancement of recovery or reorganization in the nervous system as a result of ischemic, hemorrhagic, neoplastic, degenerative, or traumatic damage comprising focally administering a composition comprising a proteolysis-enhancing agent to the central or peripheral nervous system of the subject.

34. The method of claim 33, wherein the damage is due to a stroke.

35. The method of claim 33, wherein the damage is to the spinal cord.

36. The method of claim 33, wherein the damage is to one or more nerves supplying a muscle, lung, or other organ of the body.

37. The method of claim 36, wherein the muscle is the diaphragm.

38. The method of claim 33, wherein the proteolysis-enhancing agent is selected from the group consisting of plasmin, a plasminogen activator, and an inhibitor of an endogenous plasminogen activator inhibitor.

39. The method of claim 33, wherein the agent is tissue plasminogen activator (tPA).

40. The method of claim 33, wherein the agent is plasmin.

41. The method of claim 33, wherein focal delivery is achieved by implanting a drug delivery device comprising a biocompatible polymer and the proteolysis-enhancing agent into the nervous system of the subject at or in the vicinity of an area of damage.

42. The method of claim 33, wherein the composition further comprises an agent selected from the group consisting of neurotransmitters or analogs thereof, neurally active growth factors, modulators of synaptic signaling molecules, neurally active small molecules, neurally active metals, and cells.

43. The method of claim 42, wherein the cells are stem cells.

44. A drug delivery device for implantation into the nervous system of a subject to promote recovery or reorganization in the nervous system following ischemic, hemorrhagic, neoplastic, degenerative, or traumatic damage to the nervous system, the drug delivery device comprising:
a biocompatible polymer; and
a proteolysis-enhancing agent, wherein the proteolysis-enhancing agent is released from the polymer in an amount effective to promote recovery or reorganization in the nervous system of the subject.

45. The drug delivery device of claim 44, wherein the biocompatible polymer is biodegradable.

46. The drug delivery device of claim 44, wherein the agent is plasmin, a plasminogen activator, or an inhibitor of an endogenous plasminogen activator inhibitor.

47. The drug delivery device of claim 44, wherein the agent is tissue plasminogen activator (tPA).

48. The drug delivery device of claim 44, wherein the agent is plasmin.

49. The drug delivery device of claim 44, further comprising a neural growth enhancing agent, wherein the neural growth enhancing agent is optionally selected from the group consisting of neurotransmitters or analogs thereof, neurally active growth factors, and neurally active small molecules, and neurally active metals.

50. A composition comprising a proteolysis-enhancing agent selected from the group consisting of: tissue plasminogen activator, plasmin, and inhibitors of tissue plasminogen activator inhibitors and further comprising a neural growth enhancing agent, wherein said agent is optionally selected from the group consisting of: neurotransmitters or analogs thereof, neurally active growth factors, modulators of synaptic signaling molecules, neurally active small molecules, neurally active metals, and cells.
